(19) United States
(12) Patent Application Publication
(10) Pub. No.: US 2025/0220787 A1
(43) Pub. Date: Jul. 3, 2025

(54) Headgear and Eyewear with Infrared Heating Elements

(71) Applicant: toastIR, LLC, Natick, MA (US)

(72) Inventors: Reza MOLLAAGHABABA, Natick, MA (US); Mehdi ABEDI, Brighton, MA (US); Edward Lawrence SINOFSKY, Dennis, MA (US); Levi Paul BUFFUM, Media, PA (US); Adam Holden ROSENBERG, Point Pleasant, PA (US)

(21) Appl. No.: 19/008,261

(22) Filed: Jan. 2, 2025

Related U.S. Application Data
(60) Provisional application No. 63/568,937, filed on Mar. 22, 2024.
(60) Provisional application No. 63/617,131, filed on Jan. 3, 2024.

(51) Int. Cl. H05B 6/64; H05B 6/80

(52) U.S. Cl. CPC H05B 6/6482; H05B 6/80

(57) ABSTRACT

In one aspect, an eyewear is disclosed, which includes a frame having two temples for engaging with a wearer's ears and a linking segment extending between the two temples, a screen coupled to the frame, at least one infrared radiation source coupled to the frame such that at least a portion of the infrared radiation is incident on at least a portion of the shield, and at least an infrared-reflective coating disposed on a portion of any of an inner surface and an outer surface of the protective shield for returning at least a portion of the infrared radiation incident on the shield onto the wearer's face.

### RELATED APPLICATIONS

The present application claims priority to Provisional Patent Application No. 63/617,131 titled “Headgear and eyewear with infrared heating elements,” filed on Jan. 3. 2024 and to Provisional Patent Application No. 63/568,937 titled “Headgear and eyewear with infrared heating elements,” filed on Mar. 22, 2024, both of which are herein incorporated by reference in their entirety.

### BACKGROUND

The present disclosure relates to headgears, eyewear and ear warming devices, and more particularly to headgears, eyewear, and ear warming devices that include heat-generating elements for warming a wearer's nose, ear(s), or other portions of the wearer's face.

The use of a headgear and/or an eyewear and/or earmuffs is a common practice when individuals participate in a variety of activities. For example, headgears and eyewear are employed for protection against injury in skiing, biking, and motorcycling, to name a few. Further, earmuffs are used in cold environments, including in a variety of sporting activities, such as skiing.

In many such activities, it is desirable to provide heating of a wearer's nose and/or other facial anatomical structures and/or the wearer's ears in a non-intrusive manner, e.g., in a manner that would not interfere with normal breathing of the individual wearing the headgear.

### SUMMARY

In one aspect, a headgear is disclosed, which includes a protective shell (herein also referred to as a frame), and a radiative heat-generating element (herein also referred to as a nose piece or a nose heating element or a nose warming element) configured to be mounted onto the protective shell so as to be in contact or near contact, e.g., at a separation of less than approximately 1 mm, and generally in thermal contact, with at least a portion of a wearer's nose to provide heating thereof.

In some embodiments, the heat-generating element can include a frame, and at least one infrared radiation source coupled to the frame and positioned so as to illuminate at least a portion of the wearer's nose with infrared radiation. By way of example, the infrared radiation source can generate radiation with one or more wavelengths in a range of about 700 nanometers (nm) to about 1400 nm.

An adhesive layer can be coupled to the frame for releasably attaching the frame to the wearer's nose.

In various embodiments, an electrical energy source can be coupled to the headgear, e.g., it can be incorporated in the protective shell of the headgear, for supplying electrical energy to the heat-generating element. By way of example, and without limitation, the electrical energy source can be a battery. By way of example, the shell can include a recess in which such a battery can be disposed, and a lid can optionally cover the recess.

A plurality of electrical conductors can be coupled to the protective shell for providing electrical connections between the electrical energy source and the heat-generating element. By way of example, the electrical conductors can be embedded within the protective shell.

In some embodiments, a headgear can include a switch for regulating the supply of the electrical energy to the heat-generating element, e.g., a plurality of infrared sources (such as infrared LEDs or lasers) of the heat-generating element.

Embodiments are disclosed that allow connecting and disconnecting the nose piece frame from the protective shell, which can supply electrical energy to the nose piece.

In the following discussion, the surface (face) of the nose piece that will be in proximity to or in contact with a user's skin is referred to as its front surface and the opposed surface (face) of the nose piece is referred to as its back surface. Alternatively, opposite designations may be utilized.

In some embodiments, the nose piece can be implemented as a coating of a reflective surface, a circuit containing infrared producing elements, and a front layer that is formed of a translucent or a transparent material. Such a translucent or transparent material can advantageously eliminate a direct contact of the edges of infrared sources (e.g., LEDs) with the wearer's skin, and can also in some cases modify the output angle of the radiation or its spread to better distribute the radiation onto the wearer's skin.

In a related aspect, a headgear is disclosed, which includes a protective shell and a rim extending from the protective shell. At least one light emitting diode (LED) can be incorporated into the rim and can be positioned so as to illuminate at least a portion of a wearer's face, when activated. A power source can be mounted onto the protective shell for supplying electric power to the at least one LED.

The rim can be tilted such that the radiation emitted by the at least one LED illuminates a portion of the wearer's face.

In a related aspect, a headgear is disclosed, which includes a protective shell, and any of a face shield and a visor coupled to the protective shell, and at least one LED or laser incorporated in any of the face shield and the visor and positioned so as to illuminate at least a portion of the wearer's face with infrared radiation so as to cause heating thereof.

In some embodiments, the frame of the nose-heating element can be connected to and disconnected from a connector provided on the headgear such that upon connecting the nose-heating element to the headgear (e.g., a helmet), electrical energy supplied by an energy source provided in the headgear can be provided to the nose-heating element.

In some embodiments, a nose-heating element can include a reflective back surface for reflecting at least a portion of the radiation returning from illuminated tissue back to the tissue, thereby enhancing the heating efficiency by increasing the light incident on the target tissue. By way of example, the reflective surface can be formed as an infrared-reflecting dielectric stack designed to be a high reflector at the operating radiation wavelength.

In some embodiments, a nose-heating element according to the present teachings can be in the form of a laminate having a reflective coating, a circuitry containing the infrared radiation producing elements (e.g., LEDs), and a front layer that is formed of a transparent or translucent material that allows the passage of the infrared radiation therethrough. The front layer advantageously eliminates any direct contact of the infrared radiation producing elements with the skin. Further, in various embodiments, the front layer can be shaped to modify the distribution of the infrared radiation on the skin, e.g., by focusing the radiation on certain skin areas. By way of example, the circuitry can be placed within a polymeric layer, e.g., a polyurethane or a silicone layer, which can also be formed of the same material from which the front layer is formed. In some embodiments, the back reflective layer can be a highly reflective layer.

In a related aspect, an eyewear is disclosed, which includes a frame configured to be worn by a user, a transparent optic (herein also referred to as a screen) coupled to the frame so as to be positioned in front of at least one eye of the user when the frame is worn by the user, and at least one infrared light source coupled to the optic so as to irradiate a portion of the user's face with infrared radiation. By way of example, and without limitation, the infrared light source can include one or more infrared-emitting LEDs or lasers.

In some embodiments, the infrared light source(s) can be optically coupled to the optic of the eyewear at one or both lateral surfaces thereof and the optic can include one or more surface treatment portions (e.g., textured surface portions, e.g., generated via pits or abrasions) that can interact with the radiation passing through the optic to cause scattering of at least a portion of the radiation such that at least a portion of the scattered radiation will exit the body of the optic and be incident on the wearer's skin, e.g., the wearer's nose. Alternatively, in some embodiments, a matte coating can be sprayed on the surface to affect its radiation guiding abilities.

In various of the above embodiments, the LEDs (or lasers) are separated from one another such that the radiation from the different LEDs at least partially overlap on the target tissue so as to deliver a substantially uniform radiative energy to the target tissue. By way of example, in some embodiments, the adjacent LEDs are spaced such that a separation between adjacent LEDs is substantially equal to a distance at which a radiative intensity of each LED falls to about 1/e of its maximum value.

In one aspect, an eyewear is disclosed, which includes a frame having two temples for engaging with a wearer's ears and a linking segment extending between the two temples, a screen (herein also referred to as an optic) coupled to the frame, at least one infrared radiation source coupled to the frame such that at least a portion of the infrared radiation is incident on at least a portion of the screen, and at least an infrared-reflective coating disposed on at least a portion of any of an inner surface and an outer surface of the screen for returning at least a portion of the infrared radiation incident on the screen onto the wearer's face.

In some embodiments, the screen can include a protruded section providing a recess for receiving at least a portion of a wearer's nose.

In some embodiments, the at least one infrared radiation source includes a plurality of infrared radiation sources supported by the frame of the eyewear. In some embodiments, one or more infrared radiation sources are embedded in the edge of the frame. By way of example, one or more infrared sources can be embedded into one or more temples of the eyewear and/or other portions of the frame, e.g., the bridge of the frame and/or lower portions of the frame.

In some embodiments, the one or more infrared radiation sources are configured to emit radiation with a wavelength in a range of about 800 nm to about 2 micrometers (microns), e.g., in a range of about 850 nm to about 1 micron. In various embodiments, the infrared wavelength is selected to ensure that the infrared radiation can propagate through the screen. The wavelength is chosen so as to be invisible to the wearer while still being able to propagate through common plastic materials. In some embodiments, an eye safe wavelength can be chosen. In all embodiments of the emitted light is eye safe due to the diffuse nature of this light. As noted above and discussed in more detail below, in some embodiments, a portion of the screen can include surface texturing to cause scattering of the radiation incident thereon so as to redirect at least a portion of the radiation onto the wearer's face.

A variety of infrared radiation sources can be employed in the practice of the present teachings. By way of example, the infrared radiation sources can be a light-emitting diode (LED) or an infrared laser, e.g., a diode laser.

In some embodiments, the eyewear can include a head strap that is coupled to the eyewear's frame to facilitate wearing the eyewear. A compartment can be coupled to the strap, in which an energy source, e.g., a battery, can be stored.

In a related aspect, an eyewear is disclosed, which includes a frame and a screen coupled to the frame, where the frame is configured for wearing by a user such that at least a portion of the screen is positioned in front of at least one eye of the user. At least one infrared radiation source is coupled to the frame and is configured to generate infrared radiation. At least one optical fiber coupled to the frame and in optical coupling with the infrared radiation source at a proximal end thereof can receive the infrared radiation and a radiation diffusing element coupled to the frame and optically coupled to a distal end of the optical fiber can receive at least a portion of the radiation from the optical fiber. The radiation diffusing element causes diffusion (scattering) of the radiation so as to illuminate at least a portion of a user's face.

In a related aspect, an eyewear is disclosed, which includes a frame configured to be worn by a wearer, at least one transparent optic coupled to the frame so as to allow the wearer to view an external environment through said at least one transparent optic, and an infrared nose heating element coupled to the frame so as to be positioned at least partially over the wearer's nose. The infrared nose heating element includes at least one infrared radiation source for illuminating at least a portion of the wearer's nose, thereby causing heating thereof.

In various embodiments, the infrared nose heating element can include a shell (herein also referred to as a frame) to which said at least one infrared radiation source is coupled. While in some embodiments, the infrared nose heating element is fixedly coupled to the frame, in other embodiments, it can be detachably attached to the frame.

In various embodiments, the at least one infrared radiation source is capable of emitting radiation with a wavelength in a range of about 800 nm to about 2 microns.

In various embodiments, the at least one infrared radiation source can include a plurality of infrared radiation sources incorporated in a strip attached to said shell. A variety of infrared radiation sources can be employed in the practice of the present teachings. Some examples of suitable infrared radiation sources include, without limitation, infrared light emitting diodes (LEDs), infrared lasers, among others.

In various embodiments, the at least one infrared radiation source can generate infrared radiation having a radiative power in a range of about 100 mW to about 5 W.

In various embodiments, the eyewear can further include a padding (herein also referred to as a lining or a liner) that is attached to a perimeter surface of a portion of the frame of the eyewear so as to provide a soft interface between the frame and the wearer's face. In some embodiments, e.g., when the eyewear is in the form of a goggle, the padding can provide a water-proof barrier to prevent water from entering the space between the eyewear's optic and the wearer's face. By way of example, in various embodiments, the padding can be formed of a soft polymeric material, such as a memory foam combined with a nylon mesh fabric. Further, in some embodiments, a mild adhesive can be applied onto a surface of the shell (frame) of the nose heating element to stabilize the frame on the wearer's nose.

In various embodiments, the infrared nose heating element can include an infrared-transparent thermally conductive layer to facilitate transfer of waste heat generated by electric circuitry of said at least one infrared radiation source to the wearer's nose. By way of example, the thermally conductive layer can be formed of PET (polyethylene terephthalate) or Acrylic.

The optic of the eyewear can be formed of any suitable material that is transparent to visible radiation to allow the wearer of the eyewear to view an external environment. Some examples of such materials include, without limitation, PET, polycarbonate and Trivex™, among others.

In various embodiments, the frame of the eyewear can include two temples that are configured for resting on ears of the wearer such that each of the wearer's eyes can view the external environment through a respective portion of the optic. While in some embodiments, the least one transparent optic can be a single unitary optic that can extend across at least a portion of the wearer's face, in other embodiments, the at least one optic can include a pair of optics each of which is positioned in front of one eye of the wearer when the eyewear is worn.

In a related aspect, an ear warming device, which includes at least one ear cup for positioning over an auricle of a wearer's ear, and at least one infrared radiation source positioned in said at least one ear cup for generating infrared radiation to illuminate at least a portion of said auricle and/or an ear canal of the wearer.

In various embodiments, the ear warming device includes a hanger to which said ear cup is attached, wherein said hanger is configured to be worn over the wearer's head.

In various embodiments, the ear warming device further includes a pad that is coupled to an opening side of the ear cup.

In various embodiments, the at least one infrared radiation source includes a plurality of infrared radiation sources that are disposed on a strip. In some such embodiments, the strip is disposed within the at least one ear cup.

Further understanding of various aspects of the present teachings can be obtained by reference to the following detailed description and the associated drawings, which are described briefly below.

### BRIEF DESCRIPTION OF THE DRAWINGS

FIG. 1 schematically depicts a helmet according to an embodiment having a radiant heat-generating element,

FIG. 2A is a perspective view of a nose warming element according to an embodiment of the present teachings,

FIG. 2B is a schematic view of the nose warming element of FIG. 2A coupled to a protective shell of a headgear,

FIG. 3A is a schematic view of a nose warming element according to an embodiment having a thermal sensor,

FIG. 3B is an example of a feedback circuit according to an embodiment, which can receive signals generated from the thermal sensor of the nose warming element depicted in FIG. 3A to control the de-activation of the LEDs incorporated in the nose warming element,

FIG. 4 schematically depicts a nose-heating element according to an embodiment,

FIGS. 5A, 5B, and 5C schematically depict various embodiments of a nose-heating element according to the present teachings,

FIG. 6 schematically depicts a heat-generating element according to an embodiment for use with a headgear,

FIG. 7A is a schematic nose-heating element according to an embodiment and lists examples of mechanisms for powering the nose-heating element,

FIG. 7B schematically depicts a controller that can be employed to control a thermal sensor associated with a nose-heating element and a power supply for supplying power to the LEDs of the nose-heating element,

FIGS. 8A, 8B, and 8C schematically depict a nose-heating element according to an embodiment, which can be powered via a wireless mechanism,

FIGS. 9A, 9B, 9C, 9D, 9E show various examples of headgears according to embodiments of the present teachings,

FIG. 10A shows a safety headgear according to an embodiment of the present teachings,

FIG. 10B is another schematic view of the headgear depicted in FIG. 10A,

FIG. 11 shows a headgear having a rim in which a plurality of infrared LEDs is incorporated,

FIG. 12 shows schematically a headgear having a face shield in which a plurality of LEDs is incorporated,

FIG. 13A schematically depicts an eyewear according to an embodiment,

FIG. 13B schematically depicts an eyewear according to another embodiment,

FIG. 14 is a schematic partial view of a nose-heating element according to an embodiment, where the nose-heating element includes a reflective surface,

FIG. 15 is an image of a prototype nose-heating element according to the present teachings,

FIG. 16 schematically depicts an eyewear according to an embodiment of the present teachings,

FIG. 17 is a cross-sectional view of the eyewear depicted in FIG. 16, which illustrates, among other features, an infrared reflecting coating disposed on an outer surface of the screen of the eyewear,

FIGS. 18A-18D show various examples of reflectivity of a coating applied to an outer surface of a screen of an eyewear according to various embodiments,

FIG. 19 is a schematic perspective view of an eyewear according to an embodiment having a strap to which a compartment is coupled, where a battery can be positioned in the compartment,

FIGS. 20 and 21 show the eyewear shown in FIG. 16 in a folded state,

FIG. 22 is a schematic perspective view of an eyewear according to another embodiment,

FIGS. 23A, 23B, and 23C depict various schematic views of an eyewear according to an embodiment of the present teachings,

FIG. 23D is a partial schematic view of an eyewear according to an embodiment of the present teachings,

FIG. 23E shows an eyewear according to an embodiment in which two optical fibers can transmit radiation generated by two infrared radiation sources to a radiation-diffusing element, which in turn distributes the radiation onto a wearer's face,

FIG. 23F shows an example of distribution of radiation over a portion of a user's face wearing the eyewear depicted in FIG. 23E,

FIG. 24 schematically depicts an embodiment of an eyewear according to the present teachings,

FIG. 25 schematically depicts an embodiment of an eyewear according to the present teachings,

FIG. 26 schematically depicts an eyewear according to an embodiment of the present teachings,

FIG. 27 schematically depicts an eyewear according to an embodiment of the present teachings,

FIG. 28A schematically depicts an eyewear according to an embodiment of the present teachings in which an edge-emitting source incorporated in the eyewear's frame generates radiation that is scattered via surface texturing provided over the nose section of the screen to illuminate a user's nose,

FIG. 28B is a partial schematic top view of the eyewear illustrated in FIG. 28A,

FIG. 29 schematically depicts a nose-heating system according to an embodiment of the present teachings,

FIG. 30A schematically depicts a goggle according to an embodiment of the present teachings,

FIG. 30B schematically depicts an infrared heating strip that is incorporated in the goggle of FIG. 30A,

FIG. 30C schematically depicts a perspective view of the goggle shown in FIG. 30A,

FIG. 31 schematically depicts an infrared earmuff according to an embodiment,

FIG. 32A schematically depicts a front perspective view of an eyewear according to an embodiment,

FIG. 32B schematically depicts a back perspective view of the eyewear of FIG. 32A,

FIG. 32C schematically depicts a back perspective view of an eyewear according to an embodiment, which includes a mild adhesive layer applied to a back surface of an infrared nose heating element of the eyewear,

FIG. 33A-1 is an image of a prototype of an eye wear according to an embodiment having a nose warming element,

FIG. 33A-2 is a schematic depiction of the image in FIG. 33A-1,

FIG. 33B-1 is an image of the nose heating element employed in the prototype illustrated in FIG. 33A-1,

FIG. 33B-2 is a schematic depiction of the nose heating element illustrated in FIG. 33B-1,

FIG. 33C-1 is an image of an individual wearing the prototype eyewear illustrated in FIG. 33A-1, and

FIG. 33C-2 is a schematic depiction of the image depicted in FIG. 33C-1.

### DETAILED DESCRIPTION

In one aspect, the present teachings provide headgears that include infrared radiation sources (e.g., LEDs or lasers) for heating at least a portion of the face of an individual who wears the headgear. In some embodiments, a headgear according to the present teachings can include a nose-warming element (herein also referred to as a nose-heating element) in which a plurality of LEDs (or lasers) is incorporated for coupling to the nose of the wearer for warming the wearer's nose. As discussed in more detail below, the infrared radiation can penetrate and heat the skin through its entire thickness to heat the tissue lining the nostrils. In other embodiments, a plurality of LEDs or lasers can be coupled to the rim of a headgear and positioned such that upon activation, the radiation generated by the LEDs or lasers can be incident on at least a portion of the wearer's face. In yet other embodiments, an eyewear, such as a visor coupled to a headgear, is provided that includes a plurality of LEDs or lasers such that when the visor is coupled to a respective protective shell, the radiation generated by the LEDs or lasers can illuminate at least a portion of the wearer's face to cause heating thereof.

Various terms are used herein according to their ordinary meanings in the art. The term “headgear” as used herein has its ordinary meaning and it generally refers to a covering or a protective device for the head. By way of example, a headgear can be used for safety, such as in sports, work environments, leisure activities, or military settings. Examples of such headgear include, without limitation, steel helmets, football helmets, bicycle helmets, among others. A headgear can also be in the form of a covering used for fashion, protection from the elements, etc. Examples of such headgears include, without limitation, hats, caps, etc.

The term “eyewear” is used herein to refer to an article that includes a transparent optic (herein also referred to as a screen) such that when the device is worn by a user, the transparent optic can be positioned in front of one or both eyes of a wearer such that at least a portion of light entering one or both eyes of the wearer passes through the optic before entering the eye.

The term “a radiant heat-generating element,” as used herein, refers to an element that can generate heat via emission of radiation, typically infrared radiation, and/or generation of heat associated with circuitry of one or more radiation sources, e.g., for heating of the face tissue, such as the nasal tissue. In various embodiments, a heat-generating element can generate infrared radiation, e.g., infrared radiation with a wavelength in a range of about 700 nm to about 1400 nm, such as in a range of about 850 nm to about 1000 nm, which can generate heat upon absorption by an anatomical structure.

FIG. 1 schematically depicts a headgear 100 (e.g., a helmet) according to an embodiment that includes a protective shell 102 that can be worn by a user. The protective shell can be formed of a variety of materials known in the art. In some embodiments, the shell can include multiple layers with different hardness to ensure efficient absorption of an impact. Materials suitable for fabrication of the shell are well known in the art. By way of example, a variety of polymeric materials (e.g., polyurethane), are known for fabrication of the protective shell.

The headgear 100 further includes a nose-heating element 104 that can be coupled to the protective shell and is configured for placement on a wearer's nose. As discussed in more detail below, the nose-heating element 104 includes a plurality of light emitting diodes (LEDs) that are coupled to a frame that can be placed on a user's nose.

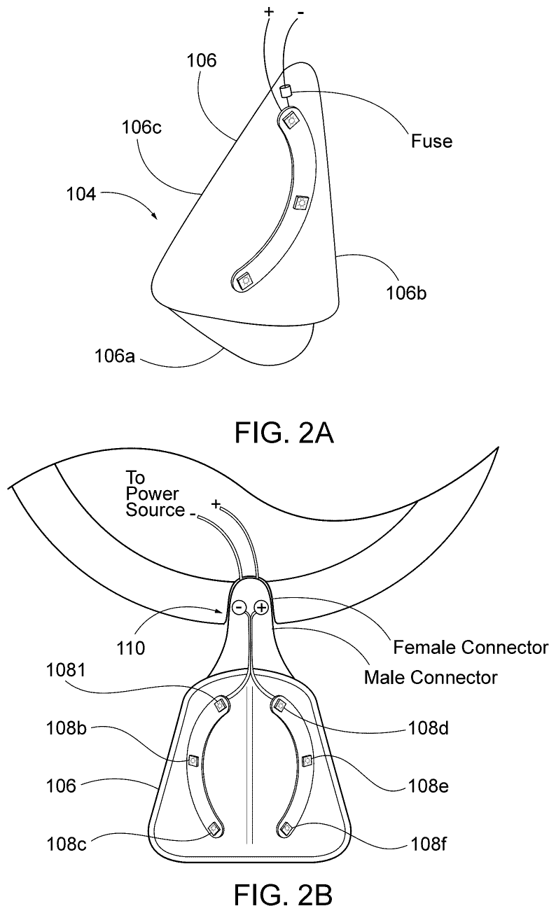

More specifically, with continued reference to FIG. 1 as well as FIGS. 2A and 2B, in this embodiment, the nose-heating element 104 includes a frame 106 in which a plurality of infrared radiation sources 108a, 108b, 108c, 108d, 108e, and 108f (herein collectively referred to as infrared radiation sources 108) are incorporated.

The nose-heating element 106 includes two wing portions 106a and 106b that are hingedly coupled via a central segment 106c, e.g., via a living hinge. In various embodiments, in use, the frame 106 can be placed on a user's nose such that the central segment is placed over the nasal bridge and the two wing portions 106a/106b can be folded about the central segment 106c to be placed over the lateral side walls of the wearer's nose. In some embodiments, the nose-heating element 104 can further include an adhesive layer to facilitate stable coupling of the nose-heating element to the user's nose.

In this embodiment, each wing portion includes three LEDs that are electrically connected in series. In particular, the LEDs 108a, 108b, and 108c, which are electrically connected in series, are incorporated in the wing portion 106a and the LEDs 108d, 108e, 108f, which are electrically connected in series, are incorporated in the wing portion 106b. Multiple groups of 3 LEDs are then connected in parallel.

The nose-heating element 106 includes a top connector portion 110 that includes a pair of male connectors that can engage with respective female connectors provided in the body of the helmet (not visible). A plurality of conductive wires electrically connects the LEDs 108 to the pair of male connectors.

With particular reference to FIG. 1 as well as FIGS. 2A and 2B, in this embodiment, the headgear includes a source of electrical power 200, e.g., a battery. By way of example, the electrical power source 200 is incorporated within a portion of the body of the headgear. By way of example, the shell 102 can include an enclosure in which the electrical power source 200 can be disposed. A plurality of electrical connectors 202 can electrically connect the power source 200 to a pair of female connectors such that upon engagement of the male connectors 110 provided in the nose-heating element with the female connectors provided in the headgear, the power source can supply electrical power to the LEDs 108. A switch 203 provided in the headgear can be used to switch the LEDs on and off. In addition to an on and an off setting, the switch can also provide other settings for adjusting the radiation intensity emitted by the LEDs to modulate the heating provided by the heat-generating element.

In some embodiments, the LEDs 108 can generate radiation in the infrared portion of the spectrum, e.g., radiation with wavelengths in a range of about 700 nm to about 1400 nm, e.g., in a range of about 800 nm to about 1 micron. In various embodiments, the infrared radiation can be chosen so as not to interfere with the user's vision. In some such cases the power source can provide an electrical power in a range of about 3 W to about 9 W, by way of example.

In various embodiments, the infrared radiation emitted by the LEDs can not only cause warming of the underlying skin, e.g., through the entire thickness of the lateral walls of the nose, but also the interior structures of the nose. Such warming of the nose can be advantageously achieved non-obtrusively, e.g., without interfering with normal breathing of an individual wearing the headgear. In various embodiments, keeping the intranasal passage warm, e.g., during activities in a cold environment, such as skiing, can lower the incidence of diseases due to cold or other rhinoviruses.

In some embodiments, a thermal sensor can be incorporated in the nose warmer, which can measure the temperature of the skin as the LEDs illuminate the nose. A feedback circuit in the headgear can receive signals generated by the thermal sensor and can switch off the transmission of power to the LEDs if the sensor's signals indicate the measured temperature exceeds a predefined threshold.

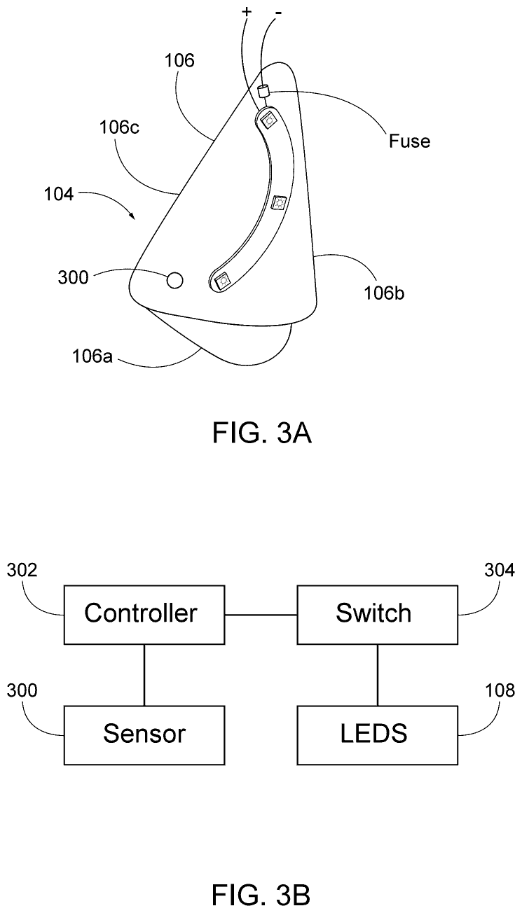

More specifically, with reference to FIGS. 3A and 3B, a thermal sensor 300 incorporated in the nose-heating element 104 can be in contact with the skin once the nose-heating element is worn by a user to measure the skin temperature. The signals generated by the thermal sensor 300 are received by a controller 302, which can send control signals to a switch 304 to activate the switch to disconnect the power source from the LEDs 108, thereby switching off the LEDs, when the signals indicate that the temperature measured by the thermal sensor exceeds a threshold.

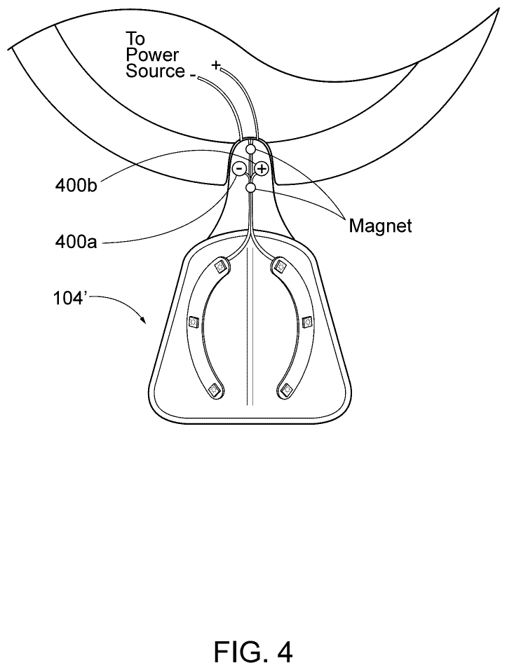

The physical coupling of a nose-heating element according to the present teachings to a headgear can be achieved using a plurality of different mechanisms. By way of example, FIG. 4 includes a nose-heating element 104′, which is similar to the nose-heating element 104 and includes a pair of magnets 400a/400b that can magnetically engage with a respective pair of magnets incorporated in the headgear (not visible in this figure) to releasably couple the nose-heating element to the headgear.

The number and/or the spatial distribution of the LEDs incorporated into a nose-heating element according to the present teachings are not limited to those utilized in the nose-heating element discussed above. In some embodiments, the number of the LEDs and their separation can be selected so as to provide a substantially uniform heating of the skin. This can be determined theoretically and/or experimentally.

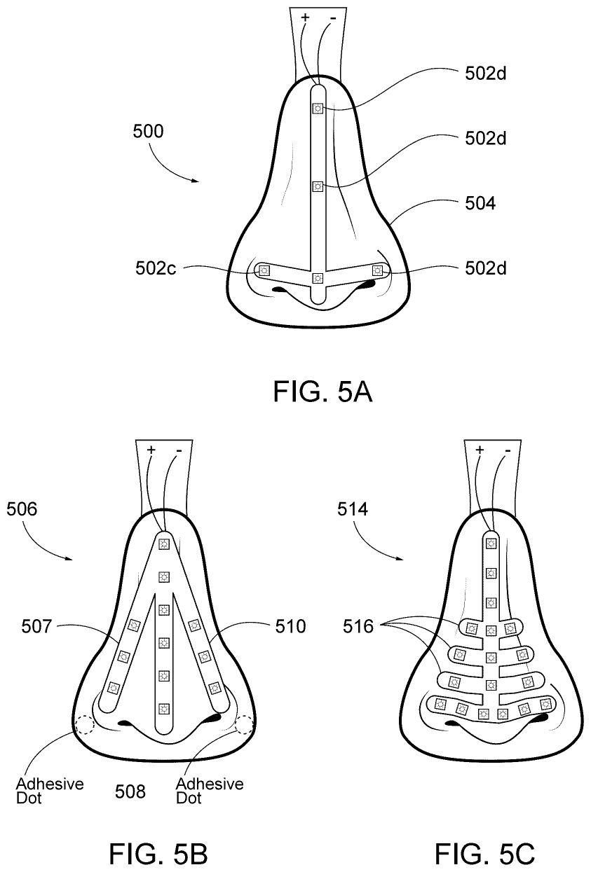

By way of example, FIG. 5A shows a nose-heating element 500 according to another embodiment, which includes five LEDs 502 incorporated in a frame 504. Three of the LEDs, namely, LEDs 502a, 502b, and 502c, extend along a central segment of the frame such that upon coupling of the frame with a user's nose, these LEDs are positioned over the nasal bridge while two other LEDs 502d and 502e are positioned on opposed lateral sides of the central segment such that upon coupling of the nose-heating element with a user's nose, each is placed over opposed lateral walls of the nose.

FIG. 5B shows another nose-heating element 506 according to an embodiment of the present teachings, which includes three LED strips 507, 508, and 510, where each of the lateral strips 507 and 510 includes three LEDs and the central strip includes five LEDs. The nose-heating element 506 further includes two adhesive dots 512a/512b for facilitating the attachment of the nose-heating element to the wearer's nose. Once coupled to a wearer's nose, the frame can be folded about the central strip such that the LEDs associated with the central strip extend along the nasal bridge, and the LEDs associated with the lateral strips 506 and 508 are placed over the lateral walls of the user's nose.

FIG. 5C shows yet another nose-heating element 514 according to an embodiment of the present teachings, which includes a plurality of LEDs 516 that are distributed according to an arrangement different than those discussed above.

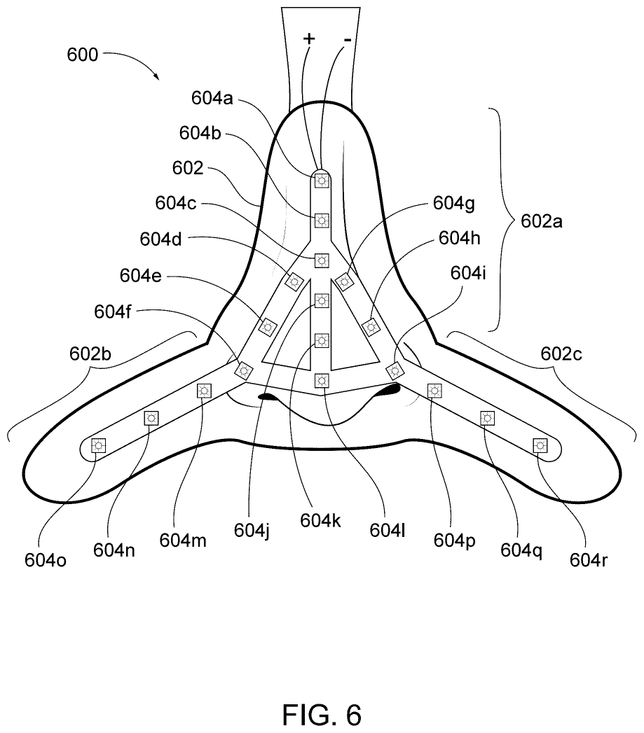

In a related aspect, a heating element is provided that includes a frame and a plurality of LEDs that are coupled to the frame for heating not only a user's nose but also other portions of a user's face. By way of example, FIG. 6 schematically depicts such a heating element 600 that includes a frame 602. The frame 602 in turn includes a nose portion 602a that can be coupled to a user's nose and two extensions 602b and 602c that are placed over a user's cheeks upon coupling of the nose portion to the user's nose. In some embodiments, the frame can include one or more adhesive layers to facilitate retaining the nose portion and/or the extensions on the user's face.

The heating element 600 includes a plurality of LEDs that are supported by the frame 600. A portion of the LEDs, such as LEDs 604a, 604b, 604c, 604d, 604e, and 604f are coupled to the nose portion of the frame can warm a user's nose and a portion of the LEDs 604g, 604h, 604i, 604j, 694k, and 604l are coupled to the extensions 602b and 602c and in use can warm the cheeks of the user.

In various embodiments, a frame of a nose-heating element according to the present teachings can be formed using a variety of suitable polymeric materials, such as polyurethane, a thermoplastic polymer, e.g., polyethylene terephthalate glycol (PETG) or acrylic. In some embodiments, the frame can be formed of a soft polymeric material that can impart sufficient flexibility to the frame such that it can be readily placed over a user's nose. By way of example, the frame can be formed of silicone.

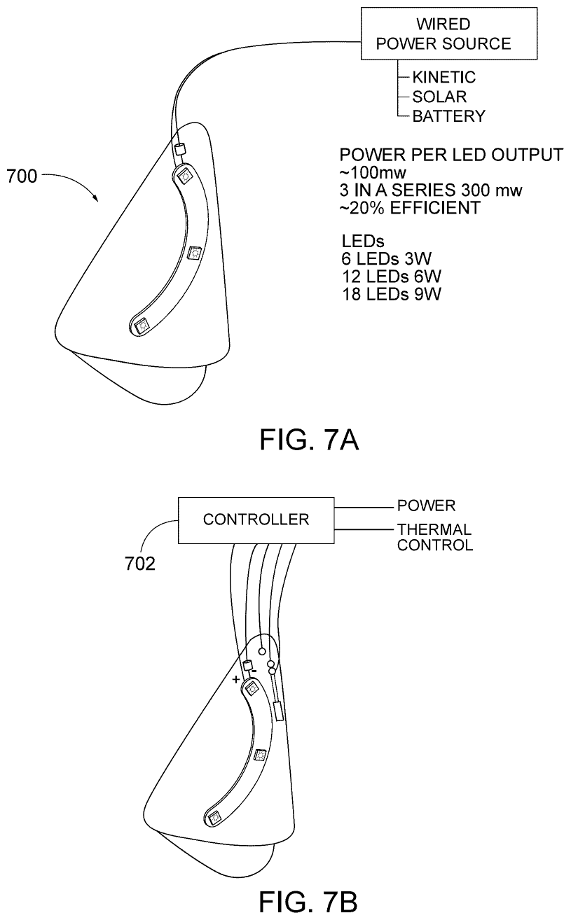

The LEDs associated with a heating element according to the present teachings can be powered in a variety of different ways. For example, wired and wireless mechanisms can be employed for powering the LEDs. By way of example, with reference to FIG. 7A, some mechanisms for supplying power to the LEDs of a nose-heating element 700 can include, without limitation, a battery, one or more solar cells incorporated in the headgear, or the kinetic energy generated by the wearer of the headgear (e.g., a bicyclist pedaling can generate rotational kinetic energy associated with the wheels that can be converted into electrical energy for powering the LEDs). By way of example, and only for illustration purposes, in some embodiments, each LED can have a radiation output power of about 100 mW generated at an efficiency of about 20%, which leads to a required electrical power of about 500 mW. In embodiments in which six, twelve, or eighteen such LEDs are utilized, the requisite electrical power can be, respectively, 3 W, 6 W, or 9 W. In various embodiments, the required power, e.g., 3 to 9 watts of power can be readily obtained using standard rechargeable batteries and is within a range that can also be supplied by a solar panel having a size similar to that of the headgear.

FIG. 7B schematically shows that a controller 702, which can be incorporated in the protective shell of a headgear, can control the operation of a thermal sensor associated with a nose-heating element and a power supply for supplying electric power to the LEDs of the nose-heating element.

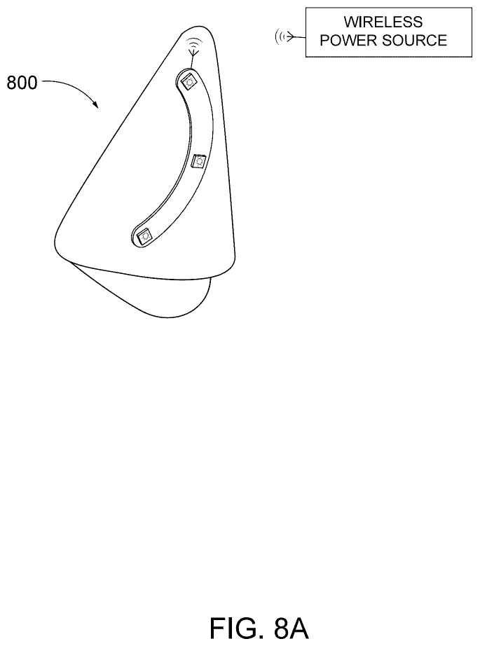

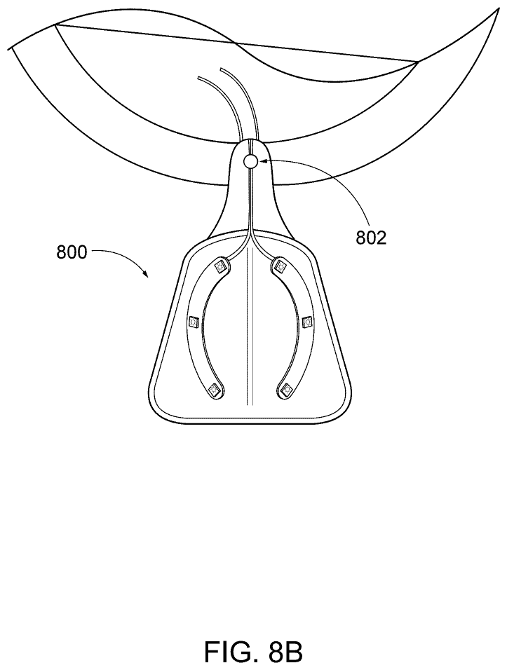

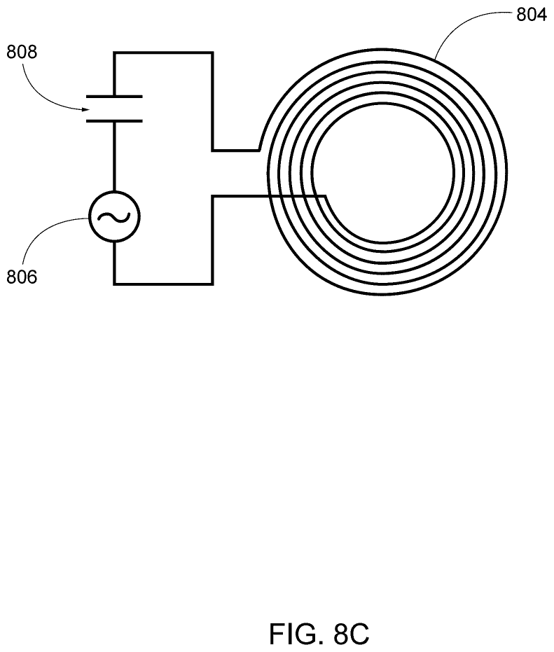

With reference to FIGS. 8A and 8B, in some embodiments, a wireless mechanism can be employed for supplying electrical power to the LEDs. For example, an induction mechanism can be employed for transfer of electrical energy to the LEDs. With reference to FIG. 8A, a nose-heating element 800 can include a coil 802 (herein also referred to as an internal coil), which is incorporated in the headgear's frame. As shown in FIG. 8B, an external coil 804 powered by an AC source 806, which is coupled to a capacitor 808, can be magnetically coupled to the internal coil to cause the generation of a current within the internal coil. More specifically, the AC source can cause the generation of an alternating current within the external coil, which in turn results in the generation of an alternating magnetic flux within the internal coil, thereby causing the generation of a current in the internal coil. A rectifier circuit as known in the art can be electrically coupled to the internal coil to rectify the induced current for application to the LED circuitry.

In some cases, the AC source and other required electrical elements, e.g., such as the capacitor 808 shown in FIG. 8B, can be incorporated into the headgear with the external coil disposed in a portion of the headgear such that it is positioned above the internal coil and in close proximity thereto. For example, as shown in FIG. 8C, the headgear can have a rim extending partially over the nasal bridge bone in which the external coil can be incorporated.

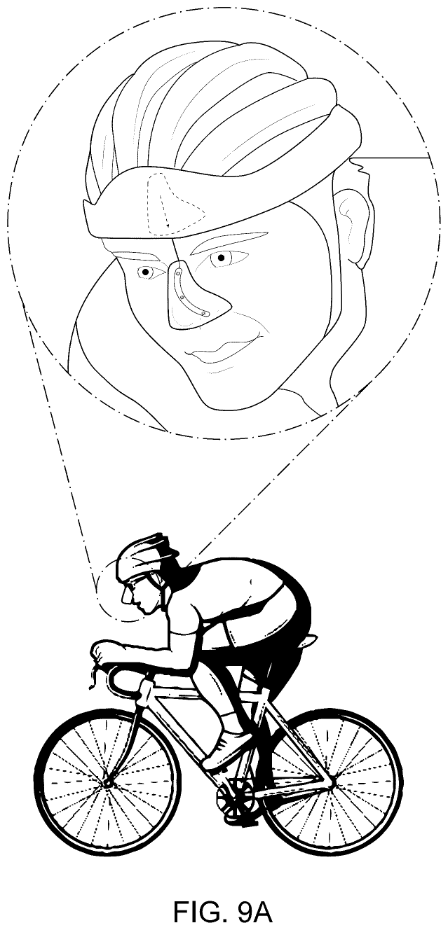

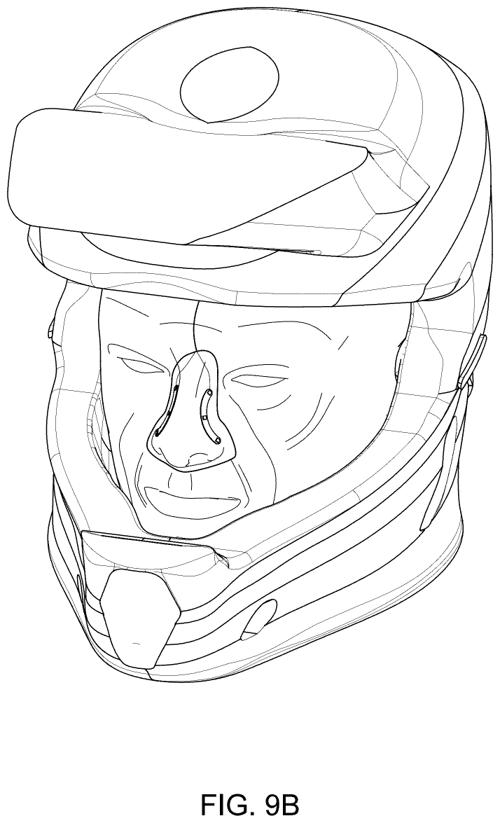

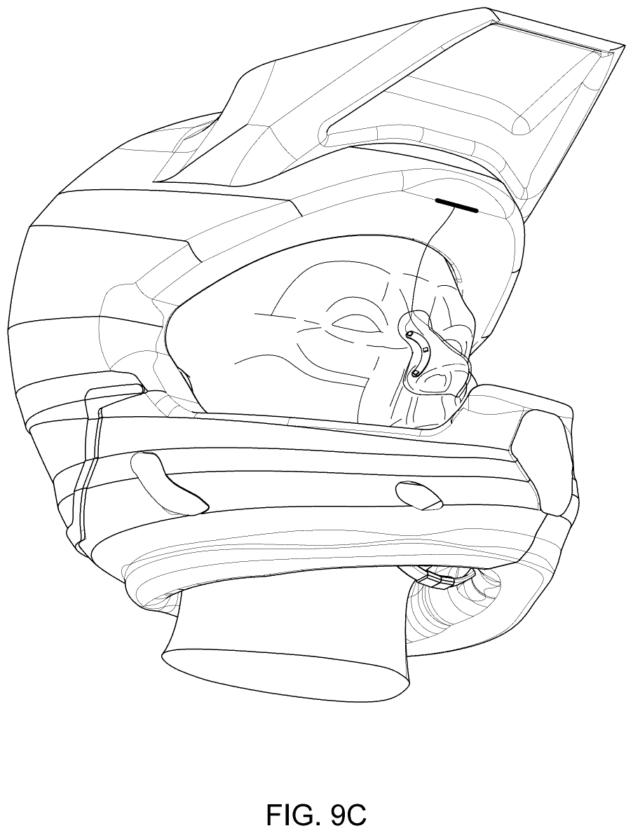

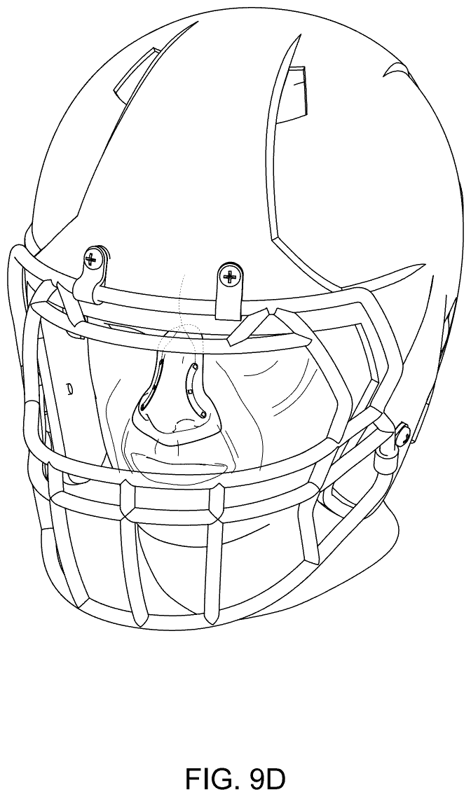

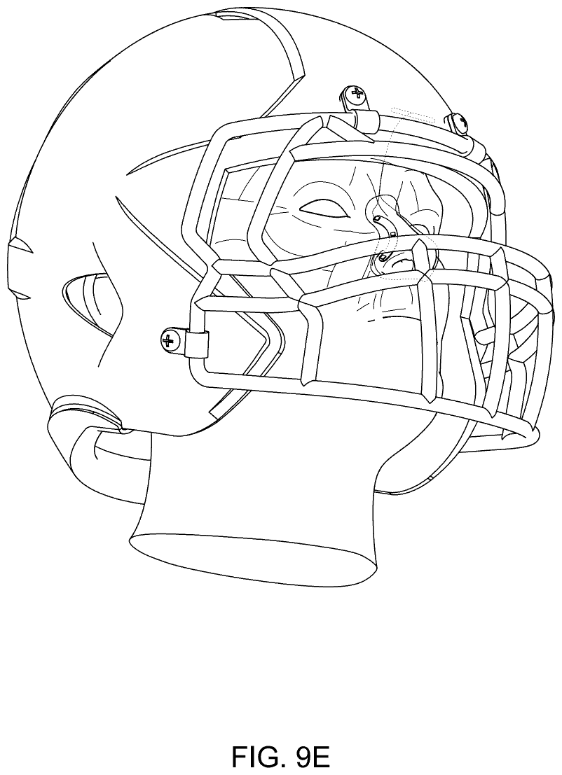

A heating element according to the present teachings can be incorporated into a variety of headgears. By way of example, a headgear according to some embodiments can be a helmet used in a variety of activities, such as skiing, biking or playing various sports (See, e.g., FIGS. 9A, 9B, 9C, 9D and 9E).

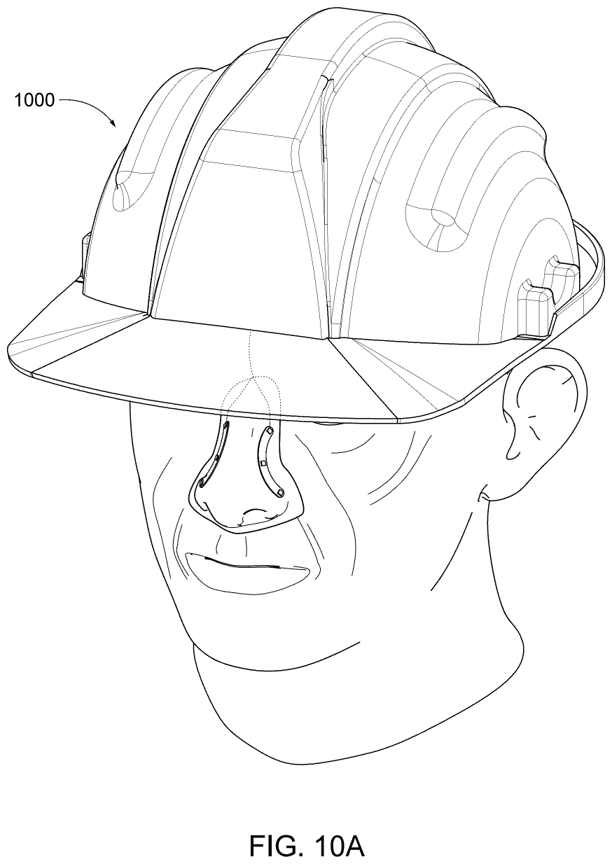

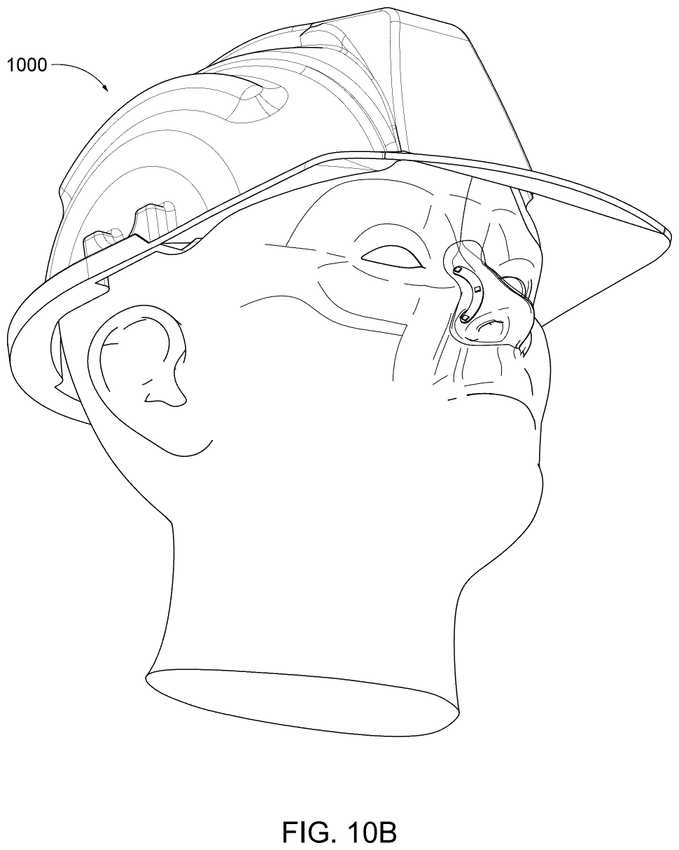

In some applications, a headgear according to the present teachings, which includes a heating element according to the present teachings, can be a safety helmet that can be used in various industries and workplaces to protect workers' heads against injury, such as in construction, manufacturing, mining, etc. FIGS. 10A and 10B schematically illustrate a safety helmet 1000 in accordance with various embodiments of the present teachings.

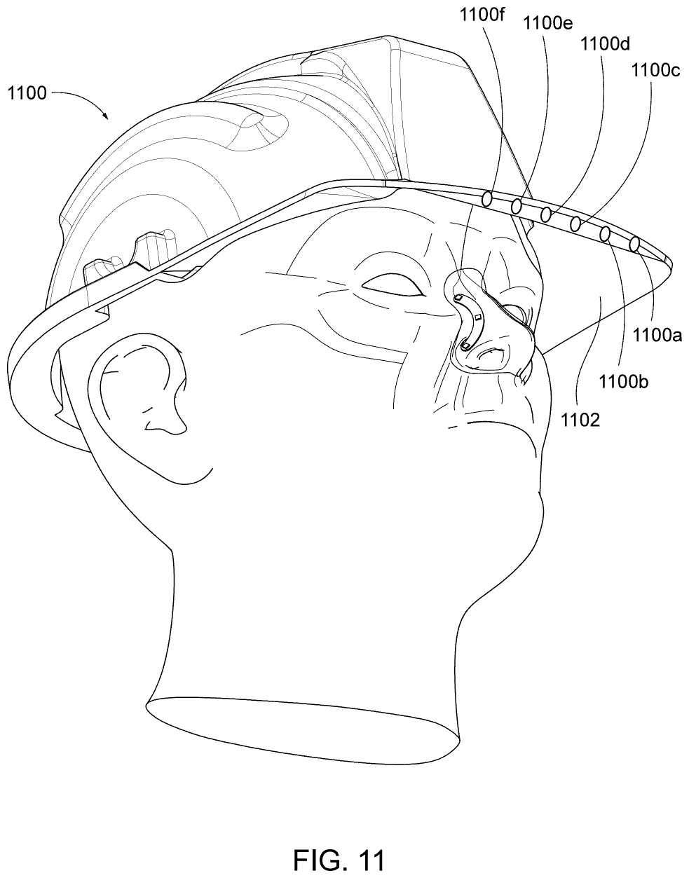

In a related aspect, a headgear is disclosed that includes one or more infrared LEDs or other suitable infrared emitting sources that are incorporated in the headgear and positioned to illuminate at least a portion of an individual's face wearing the headgear. By way of example, FIG. 11 schematically illustrates such a headgear 1100 having a rim 1102 in which a plurality of LEDs 1104a, 1104b, 1104c, 1104d, 1104e and 1104f (herein collectively referred to as LEDs 1104) is incorporated. In this embodiment, the rim is tilted and the LEDs are positioned such that their radiation-emitting surfaces can illuminate a portion of the wearer's face. By way of example, in some implementations of the headgear 1100, the emitting surfaces of the LEDs can be substantially flush with an inner surface of the headgear's rim. The LEDs can be powered in a manner similar to those described above in connection with the nose-heating element. In some embodiments, the headgear can also include a nose-heating element in addition to one or more LEDs that are incorporated in the headgear so as to illuminate the wearer's face.

In another aspect, one or more LEDs can be incorporated into a face shield or a visor associated with a headgear. More specifically, the LEDs can be incorporated into a face shield or a visor so as not to interfere with the wearer's field of view.

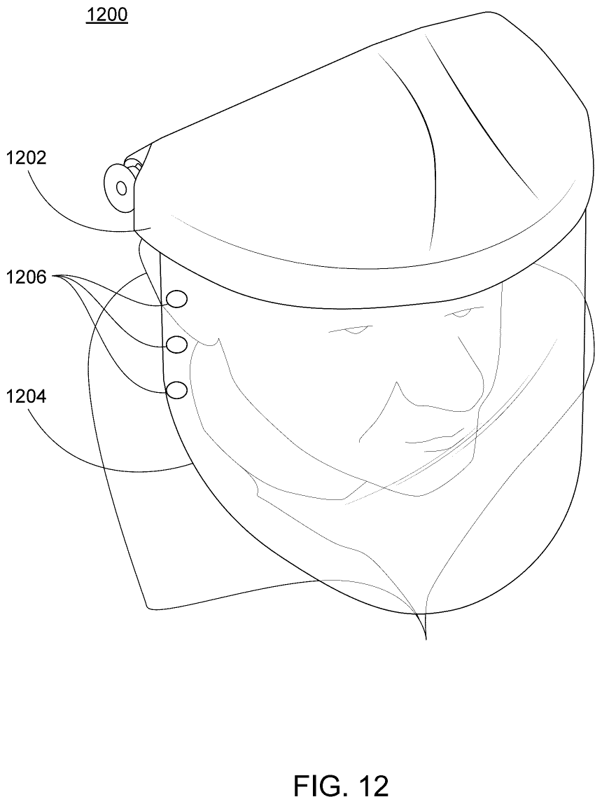

FIG. 12 schematically depicts a headgear 1200 that includes a protective shell 1202 and a face shield 1204 that is coupled, e.g., releasably, to the protective shell 1202 of the headgear. A plurality of LEDs 1206 is incorporated into the face shield in peripheral portions of the face shield so as not to block the wearer's field of view. In this implementation, the LEDs are incorporated on opposed peripheral portions of the face shield (only one set of the LEDs is visible in the figure). The natural curvature of the face shield allows the radiation emitted by the LEDs to illuminate at least a portion of the wearer's face. In some embodiments, the separation between neighboring LEDs can be selected such that the diverging radiation beams generated by the LEDs will have some overlap on the wearer's face so as to illuminate a substantially contiguous portion of the face. A power source (not shown) disposed within an enclosure provided in the protective shell can supply electric power to the LEDs via a plurality of conductors extending from the power source to the LEDs. Similar to the previous embodiments, the headgear can include a switch (not shown in the figure) to allow activating and deactivating the LEDs. Further, a thermal sensor and a feedback circuitry can be employed to deactivate the LEDs when a measured temperature (e.g., the temperature of the face shield or the visor) exceeds a predefined threshold.

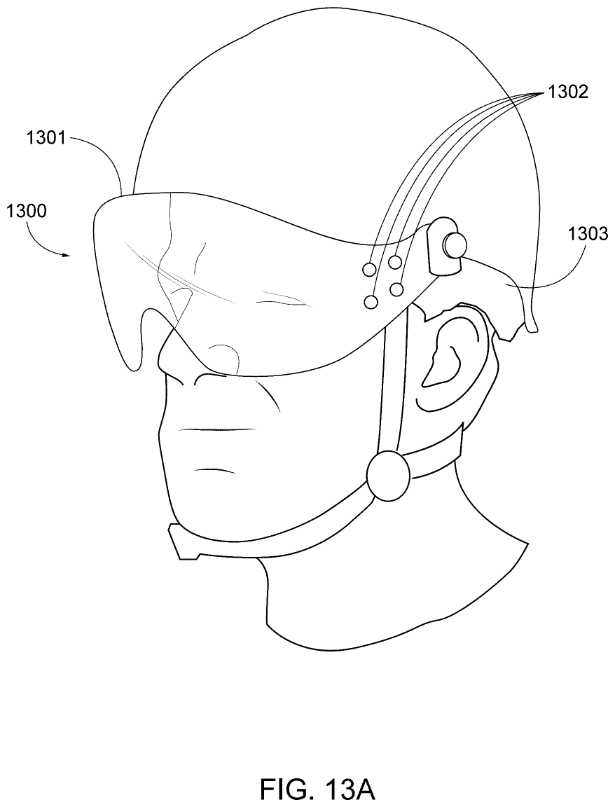

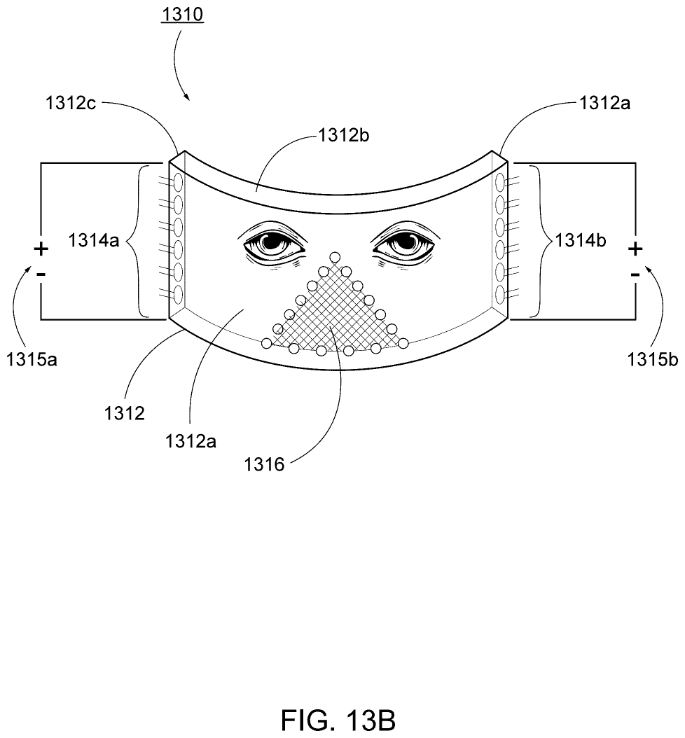

In some embodiments, one or more LEDs can be incorporated into a visor associated with a headgear so as to illuminate a portion of wearer's face. By way of example, FIG. 13A schematically depicts such an eyewear 1300, e.g., a visor associated with a helmet, having an optic 1301 in which a plurality of LEDs 1302 is incorporated. The optic is coupled to a frame 1303, which can be, e.g., a helmet worn by a user, that allows a user to wear the eyewear. The LEDs are positioned on opposed peripheral portions of the optic (only one set is shown in the figure) so as not to obstruct the wearer's field of view. While in this embodiment the LEDs 1302 are incorporated in the optic 1301, in other embodiments the LEDs 1302 and the optic 1301 can be implemented as separate units and coupled to one another, e.g., using one or more fasteners. One or more voltage sources (not shown) can supply electrical power to the LEDs.

By way of another example, FIG. 13B schematically depicts an eyewear 1310, e.g., a protective visor, according to another embodiment, which includes a transparent optic 1312 that extends between a front surface 1312a and back surface 1312b and further includes transparent lateral edges 1312c/1312d (herein also referred to as lateral surfaces) each of which can receive radiation generated by a plurality of LEDs 1314a/1314b, where the LEDs 1314a are optically coupled to one lateral edge of the optic 1312 and the LEDs 1314b are optically coupled to another lateral edge of the optic. In this embodiment, a pair of voltage sources 1315a/1315b can supply electrical power to the LEDs. While in some embodiments the LEDs can be integrated into the optic, in other embodiments the LEDs can be formed separately and be coupled to the lateral edges of the optic. Such coupling can be achieved, for example, by use of a suitable adhesive or via a plurality of pins that can be received in a set of corresponding openings provided in the lateral edges.

In various embodiments, the curved optic functions as a waveguide and may trap some of the radiation within it. Surface roughness is achieved via a plurality of surface ridges and/or pits 1316, e.g., with a height less than about 1 micron, provided on a portion of the front surface 1312a and/or a portion of the back surface 1312b of the optic can cause at least a portion of the radiation propagating through the optic to be scattered/reflected such that the radiation exits the optic to illuminate a portion of the wearer's face, e.g., the nose, and cause heating thereof. In this example, the surface ridges and/or pits are formed in a portion of the optic positioned over a wearer's nose. Optionally, a reflective layer (not shown in the figure) can cover at least a portion of any of the front and the back surface of the optic to reflect the radiation back to the wearer's face. In some embodiments, rather than or instead of using the surface treatment, such a reflective layer can be employed. Alternatively, both the surface treatment and the reflective layer may be employed for directing the heating radiation onto a target tissue.

The transparent optic 1312 can be formed of a variety of different materials. Some examples of suitable materials include, without limitation, clear plastics, glass, among others. Slightly scattered acrylics may be appropriate if the eye area is made clear. Materials like Acrylite's endlighten may be appropriate here. While in some embodiments, the optic 1312 can primarily function as a protective element, in other embodiments, in addition or instead, the optic 1312 can also provide correction of refractive errors of the wearer's eye(s).

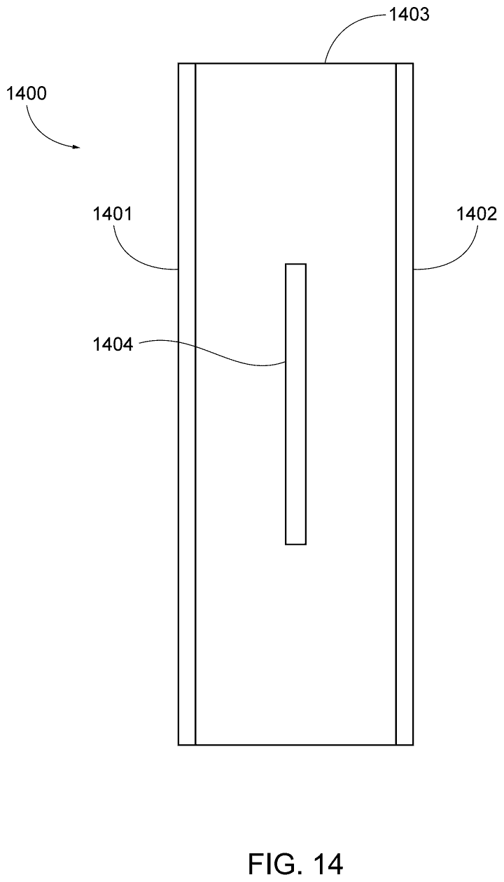

With reference to FIG. 14, in some embodiments, a nose-heating element 1400 can have a front transparent/translucent layer 1401 and a back reflective layer 1402, e.g., an infrared reflecting dielectric stack, that can retroreflect radiation that is returning from an illuminated tissue portion, thereby enhancing the heating of the illuminated portion. Such a reflecting layer may also help reduce the transfer of the generated heat to the external environment. One or more LED circuits 1404 can be incorporated in a middle layer 1403.

In some aspects, the present disclosure provides an eyewear that can provide heating of at least a portion of the face of a user wearing the eyewear.

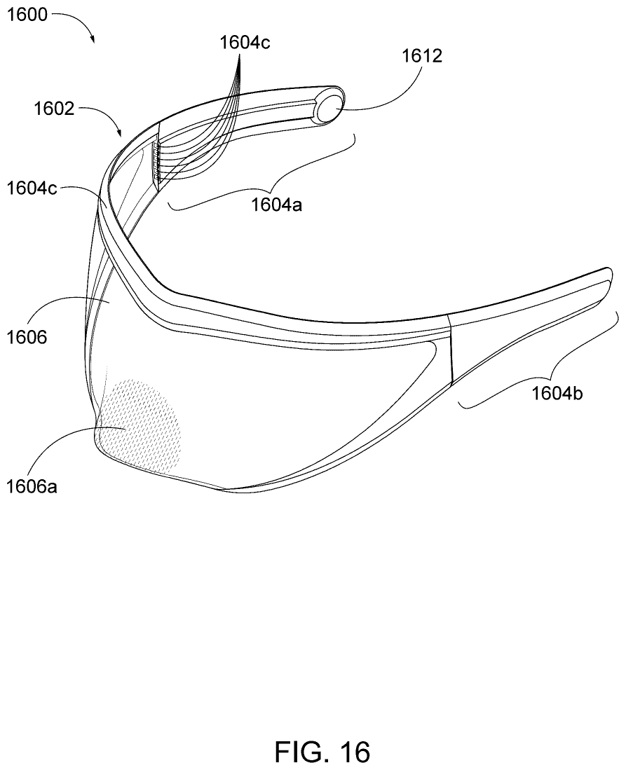

By way of example, FIG. 16 schematically depicts an eyewear 1600 that includes a frame 1602 that allows a user to wear the eyewear. The frame 1602 includes two temples 1604a/1604b and a linking segment 1604c that extends between the two temples 1604a/1604b such that the temples and the linking segment collectively and integrally form the frame 1602. In other embodiments, the temples can be detachable from the linking segment.

The eyewear 1600 further includes a substantially transparent screen 1606, which, for example, can function as a protective shield and/or a lens, that is coupled to the frame so as to cover a portion of the wearer's face including the wearer's eyes. The screen is formed of a material that is transparent to visible and near infrared. Some examples of suitable materials for forming the screen include, without limitation, a transparent plastic, such as polycarbonate, acrylic, PETG or glass.

In this embodiment, a plurality of infrared-emitting radiation sources is coupled to each of the temples to generate infrared radiation for illuminating a portion of the wearer's face. More specifically, a plurality of infrared radiation sources 1605 are supported by the temple 1604a and another plurality of radiation sources (not visible in this figure), which are similar to the radiation sources 1605, are supported by the temple 1604b. By way of example, the infrared radiation sources can be infrared light-emitting diodes (LEDs) and/or infrared lasers. While in this embodiment four such radiation sources are coupled to each temple of the frame, in other embodiments the number of the radiation sources can be different.

By way of example, the infrared-emitting radiation sources can emit infrared radiation with a wavelength in a range of about 800 nm to about 2 microns, by way of example. In some embodiments, a wavelength of 850 nm may be chosen as it is invisible to the eye.

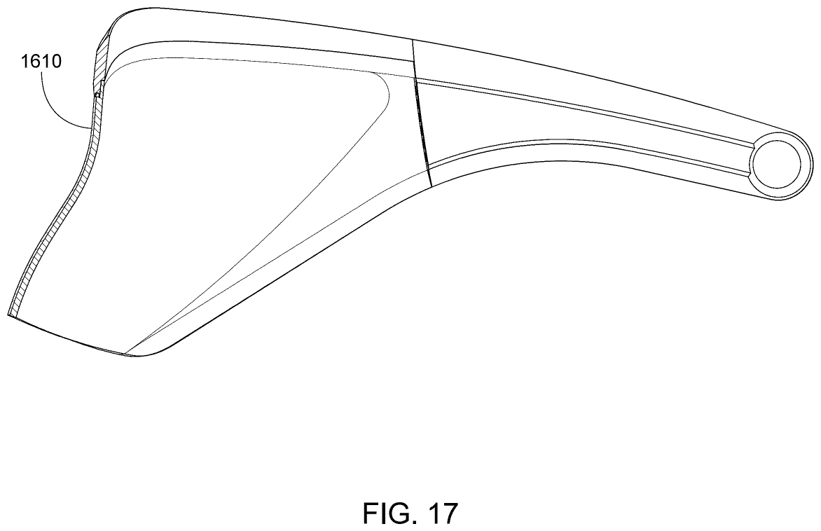

At least a portion of the radiation emitted by the infrared sources can directly illuminate the wearer's facial tissue and cause warming thereof. Further, with continued reference to FIG. 16 as well as FIG. 17, at least a portion of the infrared radiation emitted by the infrared sources can illuminate a thin infrared-reflecting film 1610 deposited on an outer surface of the screen and be reflected by the reflective film to be directed back to the wearer's face, and particularly to the portion of the wearer's face covered by the screen. By way of example, the infrared-reflecting coating can have a thickness in a range of about 100 mm to about 200 mm. The reflectors are typically known as dielectric reflective stacks. The infrared-reflecting coating can be formed, e.g., of a mixture of titanium oxide, zinc oxide, zinc sulfide, kaolin, aluminum oxide, and inorganic hollow microspheres. The reflected infrared radiation can impinge on the skin tissue and cause warming thereof, e.g., in a manner discussed above in connection with the previous embodiments.

In this embodiment, the screen includes a protruded section 1606a that provides a cavity (herein also referred to as a recess) for receiving at least a portion of the wearer's nose. A portion of the infrared radiation reflected by the infrared-reflecting coating can be incident on the wearer's nasal tissue to cause its warming. In particular, in various embodiments, at least a portion of the generated infrared radiation can enter the screen and be incident on the infrared reflecting coating, which can reflect at least a portion of the infrared radiation onto the wearer's face, e.g., a portion of the wearer's face including the nose. The infrared radiation incident on the face can cause warming of the facial tissue. For example, the reflected infrared radiation, or at least a portion thereof, can penetrate through the outer nasal tissues and hence contribute to the warming of the mucosal tissue of the nasal cavities.

In some embodiments, the spacing of the adjacent radiation sources is selected such that the location of the 1/e reduction in power emitted by each source overlaps with the location of the 1/e reduction in power emitted by an adjacent source as a function of distance from the center of each source. The power contributed by each infrared source at the 1/e boundary is about 37% of the maximum power. If two adjacent infrared sources are spaced by about two times the distance associated with the 1/e reduction in power, then two 1/e boundaries overlap. Thus, the combined power where the 1/e boundaries meet between two adjacent sources is about +37%, i.e., about 75% of the maximum power deliverable to a target surface (e.g., facial tissue, or the infrared-reflecting coating) that is closest to each source. In various embodiments, the cumulative effect of this preferred spacing is the delivery of a substantially uniform radiation intensity to the infrared-illuminated portions of the wearer's face. Further details regarding optimal spacing between the infrared sources can be obtained in U.S. Published Patent Application No. 20210252306 entitled “Methods and devices for infrared therapy” (now U.S. Pat. No. 12,053,642), which is herein incorporated by reference in its entirety.

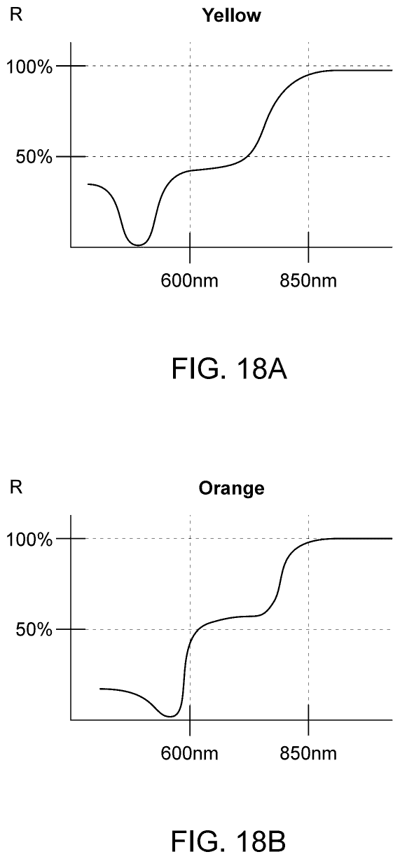

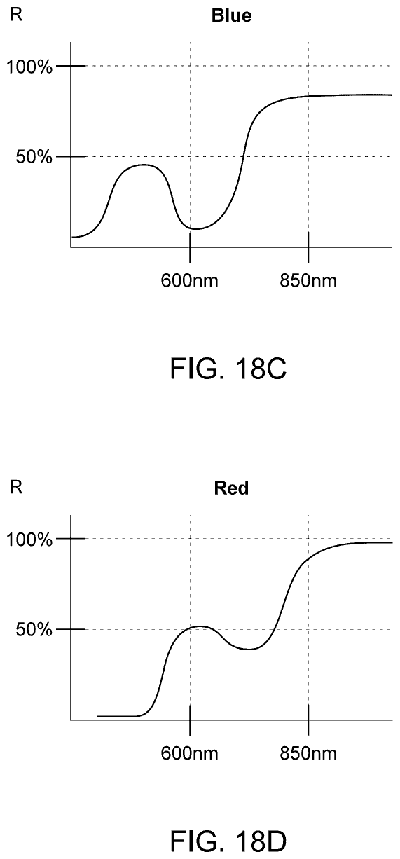

In some embodiments, the infrared-reflecting coating can also be configured to concurrently provide some reflection of light at a visible wavelength so as to reflect a portion of the external light incident on the screen, thereby imparting a desired color of the screen to an external observer in a sunglass light effect. In other words, in some embodiments, the eyewear can be in the form of a sunglass shade with high reflection at the color of the infrared sources. By way of example, FIG. 18A shows an example of the spectral reflection of a coating that provides reflection of both infrared radiation as well as radiation in the visible portion of the spectrum such that the screen appears as yellow to an observer. FIGS. 18B, 18C and 18D present other examples of the reflection spectrum of a coating that can be applied to the inner and/or outer surface of the screen so as to provide, an orange, a blue and a red appearance to the eyewear while concurrently reflect the incident infrared radiation.

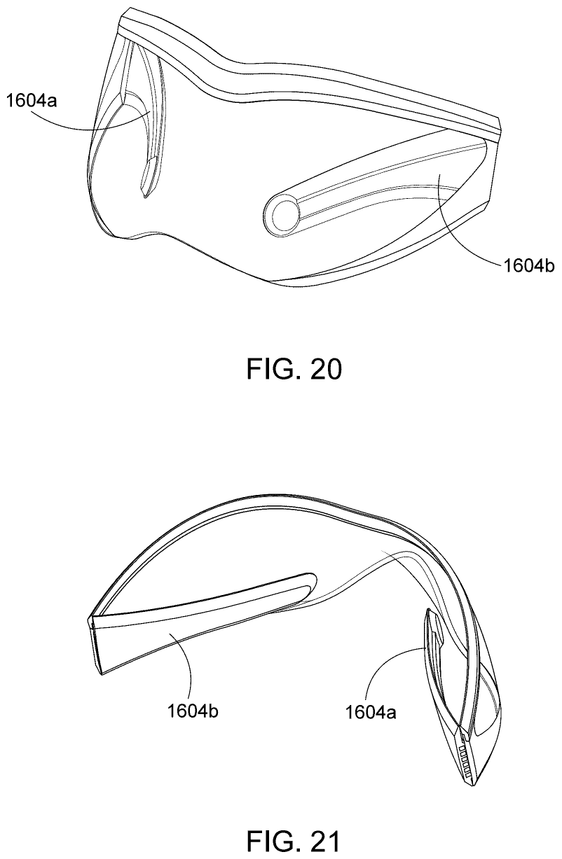

Referring again to FIG. 16, a touch sensor 1612 incorporated in the frame 1602 and associated switching circuitry (not shown in this figure) that can be incorporated, e.g., within the temple 1604a to allow a user to activate and deactivate the infrared sources. In addition, in this embodiment, the temples 1604a and 1604b are foldable, e.g., to provide a more convenient storage of the eyewear when not in use. With reference to FIGS. 20 and 21, in this embodiment, the folding of the foldable temples results in the deactivation of the infrared sources. Upon unfolding of the temples, the infrared sources can be activated via the touch sensor 1612. As noted above, the foldable design allows for easy compact storage of the eyewear.

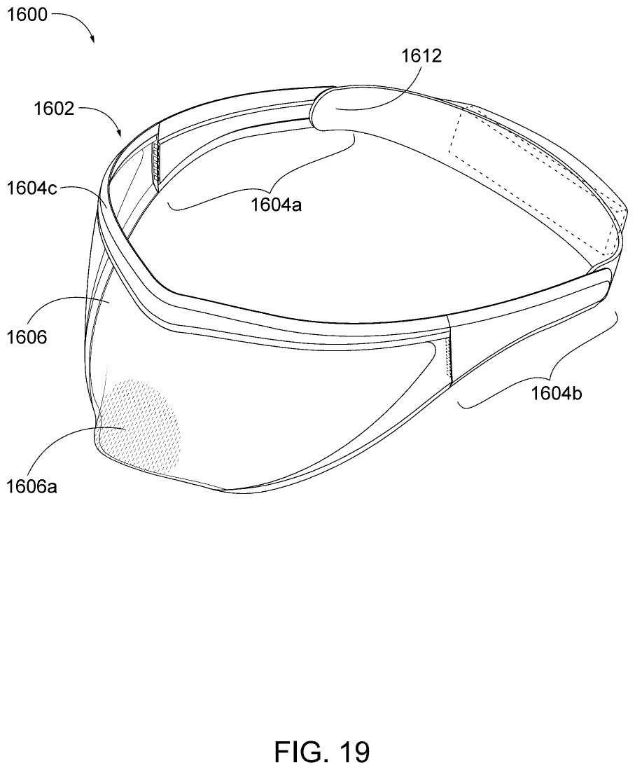

With reference to FIG. 19, the eyewear 1600 can include a head strap 1900 that can be releasably coupled to the frame 1602 and can include a compartment 1902 in which a battery 1904 suitable for powering the infrared sources can be stored. In other embodiments, one or more batteries can be stored in one or more compartments provided in one or both temples of the frame. By way of example, the battery can be a 5-volt battery provide 10 W of power, though other types of batteries can also be employed depending in the requirements of a specific embodiment. A plurality of conductive wires (not visible in this figure) can connect the battery to the infrared sources upon engagement of the strap with the frame.

In other embodiments, the eyewear can be a visor associated with a helmet, where the power source for energizing the infrared sources is incorporated in the helmet, e.g., in a manner discussed above.

The eyewear 1600 can be utilized in a variety of activities. For example, the eyewear 1600 can be used as a biking goggle, or can be utilized for skiing, among other applications.

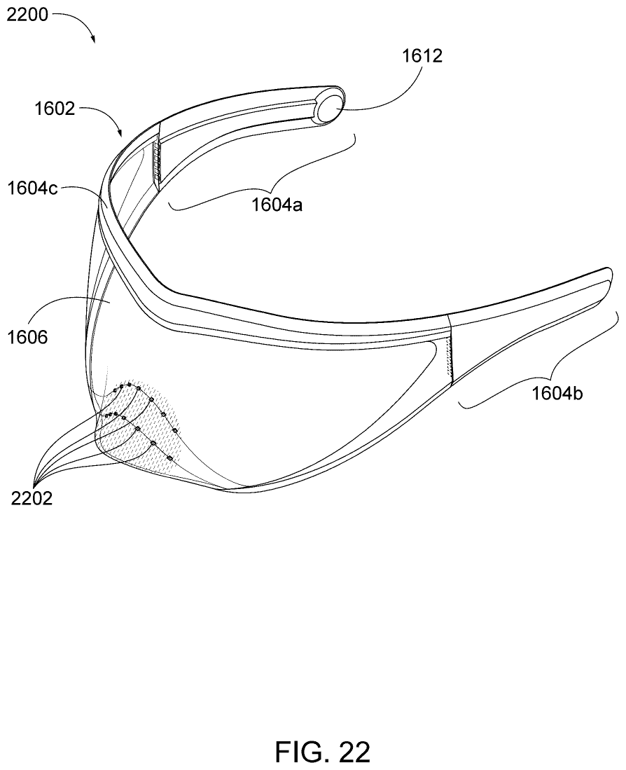

FIG. 22 schematically depicts an eyewear 2200 according to another embodiment, which instead of or in addition to the infrared radiation sources coupled to one or both temples of the eyewear's frame, includes a plurality of smalls LEDs 2202 with micro circuitry that are positioned on the inner surface of the screen. By way of example, the LEDs can have a dimension of about 300 microns by 300 microns and can be invisible to the user of the eyewear. The microcircuitry should be barely visible through the outer reflective surface. In this implementation, the LEDs 2202 are positioned along two rows over the protrusion provided in the screen for receiving at least a portion of a wearer's nose. By way of example, the number of LEDs can be in a range of about 9 to about 12, with each LED generating infrared radiation at a power lever of about 120 mW.

In other embodiments, the LEDs 2202 can be positioned between the reflective coating and the outer surface of the screen in other areas of the screen, e.g., closer to one or more of the boundaries between the screen and the frame.

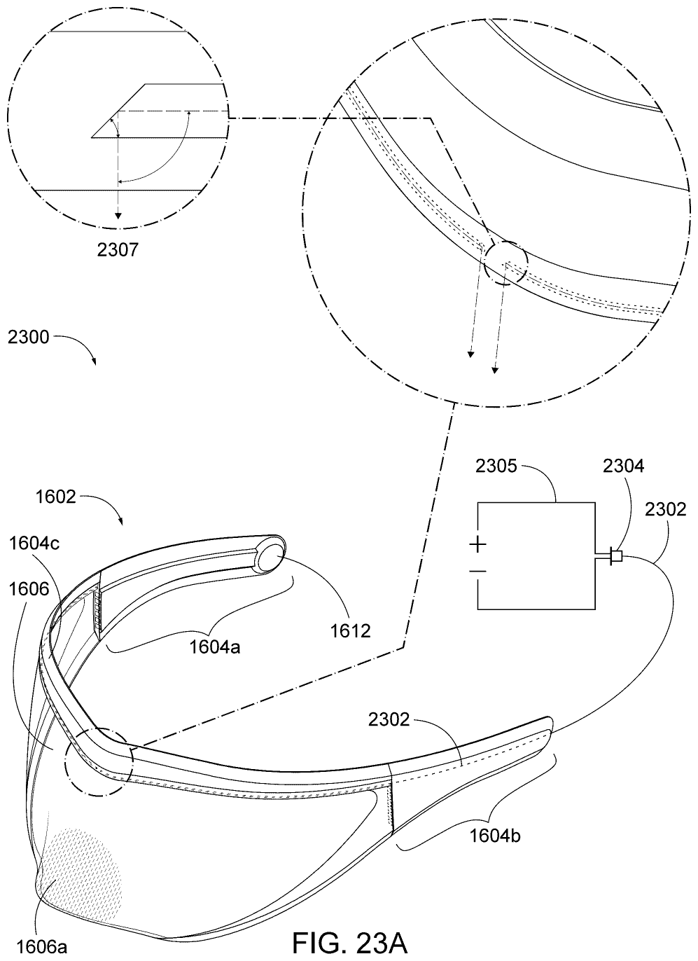

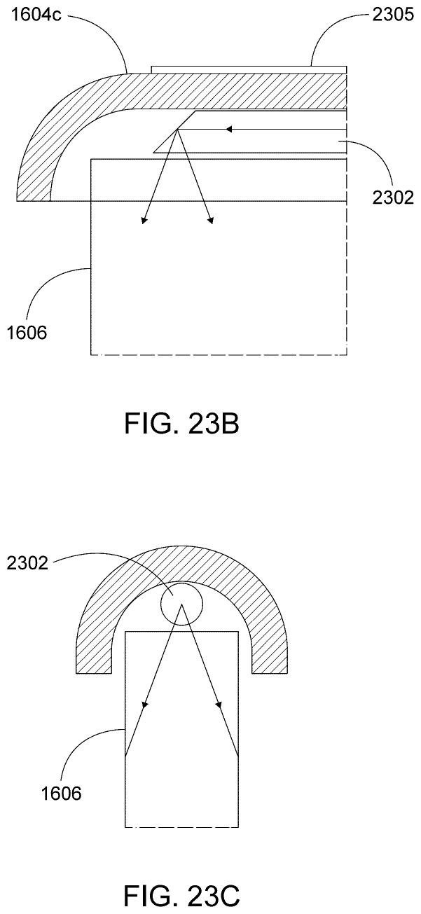

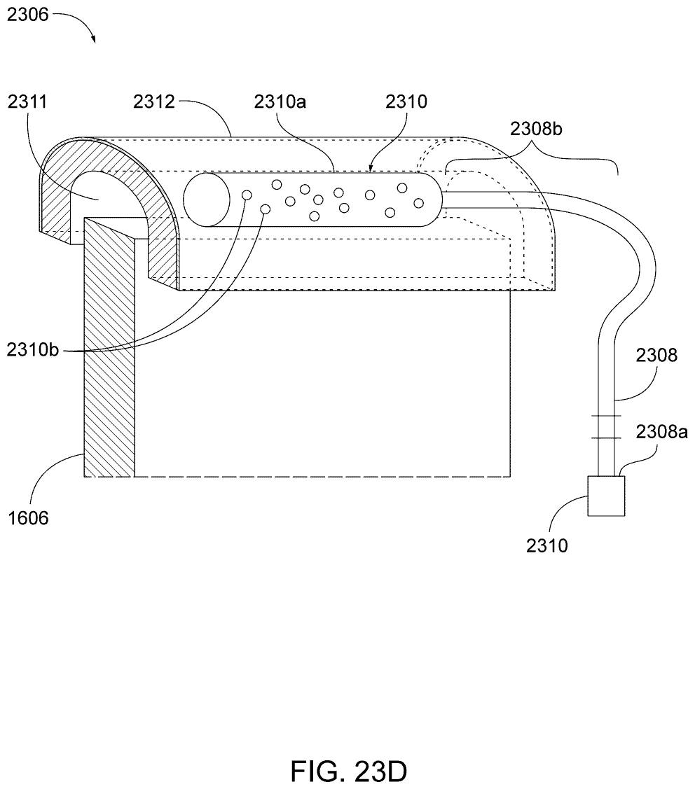

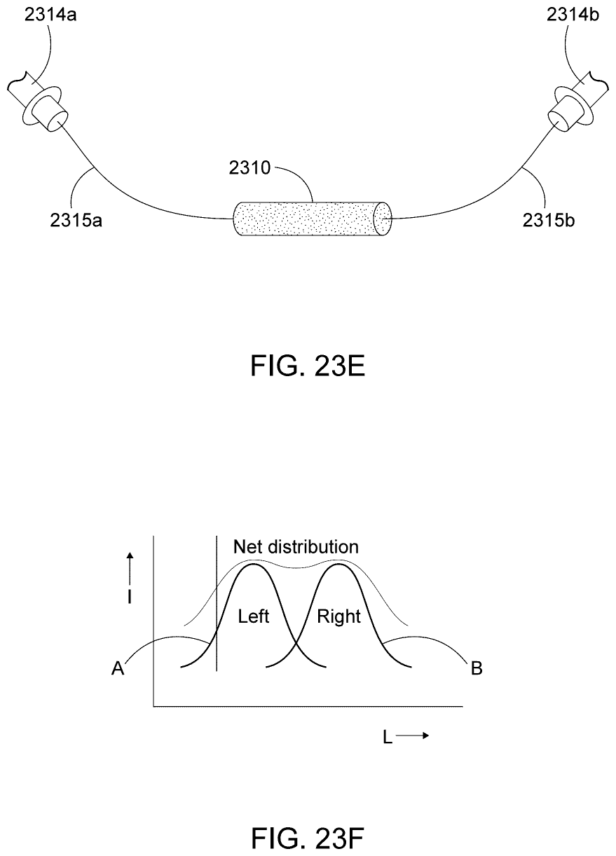

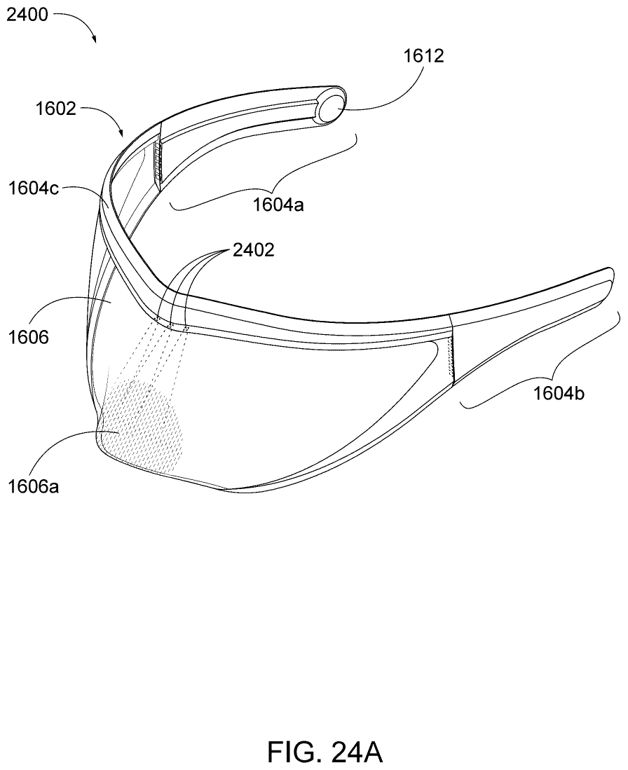

With reference to FIGS. 23A, 23B, and 23C, an eyewear 2300 according to another embodiment includes, instead of or in addition to the infrared sources positioned in one or both temples of the eyewear, a side-emitting optical fiber 2302 that extends from a proximal end that is optically coupled to an infrared radiation source 2304, powered by a battery 2305, into a channel formed in the bridge of the eyewear's frame such that a chiseled distal end of 2307 of the optical fiber 2302 is positioned over a user's nose bridge and emits the radiation received from the radiation source into the screen 1606 of the eyewear in a direction that is substantially perpendicular to the longitudinal axis of the optical fiber. This is known as edge lighting. The screen, which may be formed, e.g., of polycarbonate or acrylic, can act as a waveguide, in combination with the infrared reflecting coating 2305 deposited on the outer surface of the screen to direct the light to the nose section 1606a. In some embodiments, surface texturing on the outer and/or the inner surface of the screen can scatter at least a portion of the radiation incident thereon so as to redirect at least a portion of the scattered radiation onto a user's face.

In some embodiments, a portion of the upper section 1604c of the frame above the chiseled face of the optical fiber can be coated with an infrared reflective coating to redirect radiation that may be directed in a direction away from the screen 1606. In some embodiments, the battery 2305 can be stored in a compartment provided by a strap attached to the eyewear. In other embodiments, the battery 2305 can be stored in a compartment formed in a helmet to which the eyewear can be coupled. The light emitted into the waveguide propagates down until it hits the surface treatment, which spoils the waveguiding condition causing the light to exit and interact with the user's nose.

FIG. 23D schematically depicts a partial view of an eyewear 2306 according to another embodiment, which includes an optical fiber 2308 that receives radiation from a radiation source 2310 at a proximal end 2308a thereof. By way of example, the radiation source can be a laser diode generating 2 W of power, though other types of infrared radiation sources can also be employed. In some embodiments, the laser diode and its associated circuity can be stored, within an enclosure provided in a head strap to which the eyewear can be coupled, or alternatively, within an enclosure provided in a helmet to which the eyewear can be coupled.

A distal portion 2308b of the optical fiber 2308 is incorporated into the upper rim of the frame of the eyewear with the distal end of the optical fiber is optically coupled to a radiation-diffusing element 2310, which is incorporated in the bridge section of the eyewear's frame, which in turn diffuses the radiation received from the optical fiber such that at least a portion of the radiation is directed into a screen 1606 of the eyewear and is at least partially reflected by an infrared reflecting coating deposited on an outer surface of the screen onto a wearer's face (not visible in this figure). In some embodiments, surface texturing provided on a portion of the screen's outer and/or inner surface can scatter at least a portion of the radiation onto a wearer's face.

By way of example, the radiation-diffusing element 2310 can include a matrix 2310a in which a plurality of scattering centers 2310b is distributed. A suitable radiation diffusing element that can be used in various embodiment as the radiation diffusing element 2310 is disclosed in U.S. Pat. No. 5,632,676 titled “Loop diffusers for diffusion of optical radiation,” and U.S. Pat. No. 6,270,492 titled “Phototherapeutic apparatus with diffusive tip assembly,” both of which are herein incorporated by reference in its entirety.

With continued reference to FIG. 23D, in this embodiment, the radiation diffusing element 2310 as well as the distal portion 2308a of the optical fiber 2308 are positioned in a channel 2311 provided in the upper portion of the eyewear's frame such that the radiation diffusing element 2310 would be positioned over a wearer's nose bridge when wearing the eyewear. In this embodiment, an infrared reflective coating 2312 can be deposited on an at least an upper portion of the inner surface of the channel and/or on at least on a portion of the upper outer surface of the frame associated with the portion of the channel in which the radiation diffusing element is positioned.

FIG. 23E shows that in another embodiment, two infrared laser sources 2314a/2314b are employed to deliver the infrared radiation to two optical fibers 2315a and 2315b each of which is coupled to one end of the radiation-diffusing element 2310. The use of the infrared laser sources 2314a/2314b for delivering radiation to the radiation-diffusing element can advantageously result in a more uniform illumination of the wearer's face. By way of example, FIG. 23F depicts a graph A showing an example of an angular distribution of the radiation intensity illuminating a wearer's face as a result of the radiation supplied by the laser 2314a, and a graph B showing an example of an angular distribution of radiation intensity illuminating a wearer's face as a result of the radiation supplied by laser 2314b.

Similar to the embodiment depicted in FIG. 23D, portions of the optical fibers as well as the radiation-diffusing element are disposed in a channel formed in the upper section of the frame (See, channel 2311). With both lasers activated, the intensity distributions partially overlap to provide a substantially uniform illumination intensity on the wearer's face. The channel in which portions of the optical fiber and the radiation-diffusing element are disposed, such as channel 2311, can also function as a heat sink, e.g., for removing heat from the radiation diffusing element. By way of example, a metal strip can run along the length of the channel in which the radiation diffusing element and the distal portion of the optical fiber are disposed to help dissipate the generated heat. Moreover, in some embodiments, the circuitry of the diode laser can be attached to the heat sink, e.g., a thin metal plate, that is coupled to the end of the frame so as to be in thermal contact with the laser circuitry and help dissipate the heat generated by that circuitry.

FIG. 24 schematically depicts an eyewear 2400 according to another embodiment, which similar to the previous embodiments includes a frame 1602 and a screen 1604 coupled to the frame. In this embodiment, instead of or in addition to the infrared sources coupled to frame's temples, a plurality of edge-emitting LEDs (3 LEDs in this implementation) 2402 is coupled to the bridge portion of the frame to generate infrared radiation for warming the wearer's facial tissue. In some embodiments, the LEDs (or laser diodes) 2402 can be embedded in the bridge portion of the frame or be coupled to a surface portion of the frame. In some implementations, the power consumption of the LEDs 2402 can be about 1 W. The LEDs can be powered in a manner discussed above. In this embodiment the bridge has heat dissipative properties to cool the LEDs or lasers.

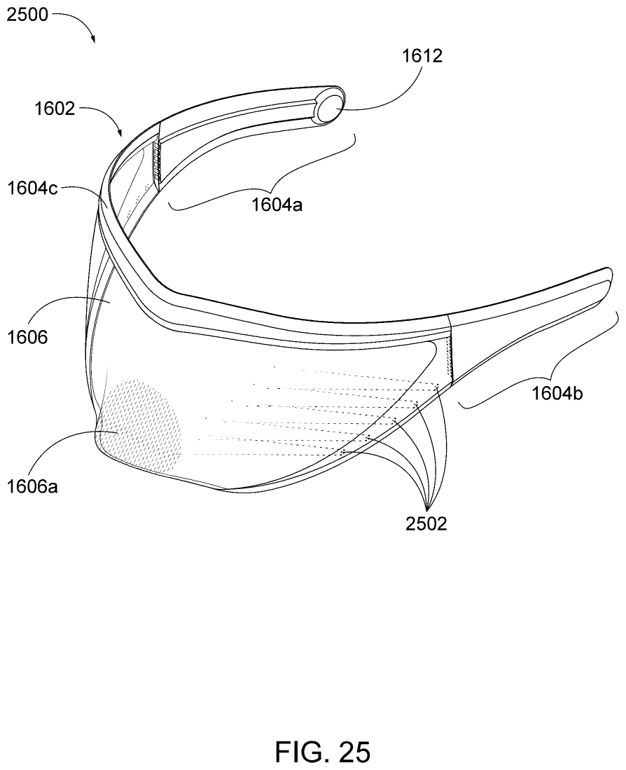

FIG. 25 schematically depicts an eyewear 2500 according to another embodiment, which includes a frame 1602 to which a screen 1606 is coupled. A plurality of edge-emitting LEDs 2502 can be coupled to a lower portion of the frame for illumination of the wearer's facial tissue. The radiation generated by the edge-emitting LEDs 2502 can propagate through the screen and be reflected via an infrared reflective coating deposited on the outer surface of the screen (not visible in this figure) onto a wearer's face to cause warming thereof.

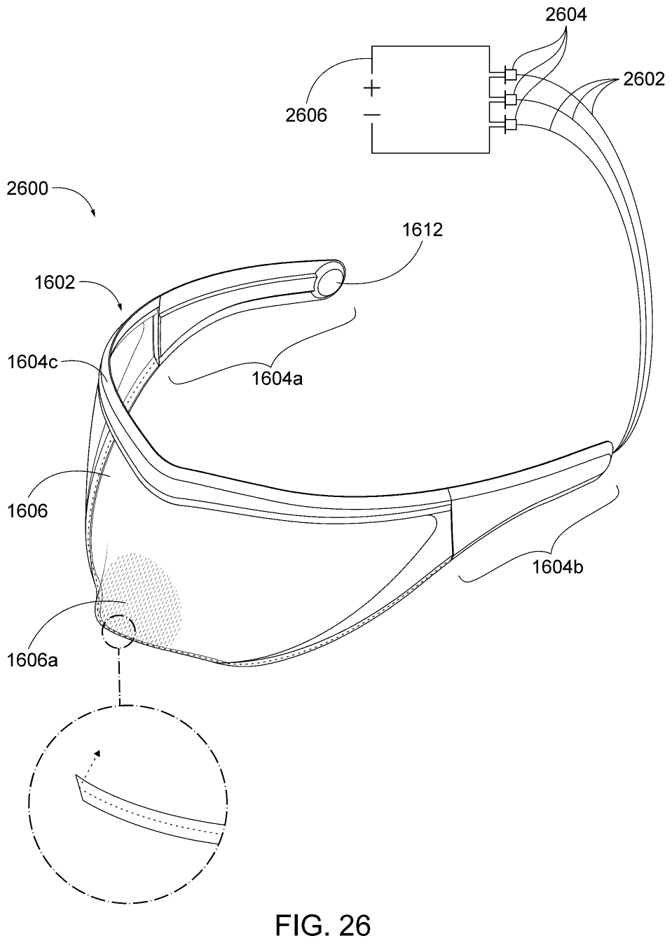

FIG. 26 schematically depicts an eyewear 2600 that includes a frame 1602 and a screen 1606 coupled to the frame. A plurality of side-emitting optical fibers 2602 is coupled to the frame. The side-emitting optical fibers 2602 can be embedded in the frame or can be positioned on an inner surface of the frame and covered with an infrared transparent cover. In this embodiment, a plurality of LEDs (or laser diodes) 2604 can supply radiation to the plurality of optical fibers. In some embodiments, the LEDs 2604 can be positioned on a heat sink (not shown) that is in turn positioned on a top surface of a battery 2606 that supplies electrical power to the LEDs. In some implementations, the LEDs 2604 and the battery 2606 can be stored in a compartment coupled to a head strap or a helmet to which the eyewear can be coupled.

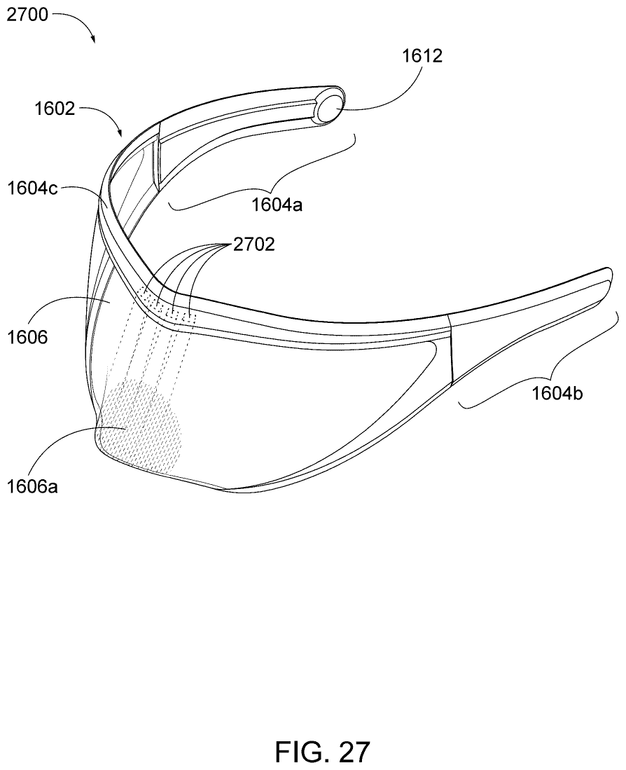

In some embodiments of the eyewear, in addition to or rather than incorporating the infrared sources in one or more temples of the frame, the infrared sources can be distributed over other portions of the frame. By way of example, FIG. 27 schematically depicts an eyewear 2700 that includes a frame 1602 and a screen 1606 that is coupled to the frame. In this embodiment, a heat sink channel, such as the channel 2311 depicted in FIG. 23D, can be formed in a portion of the frame, in this case, in the bridge portion of the frame, and a plurality of diode lasers 2702 can be surface mounted into the heat sink channel. The heat sink channel can be formed via forming a channel in the frame and depositing a metal on the lower surface (and in some cases also on the lateral surfaces) of the channel. The radiation generated by the diode laser 2702 can propagate through the screen and is emitted in the area of surface treatment. The emitted light is further reflected by a reflected coating deposited on an outer surface of the screen to be redirected onto the wearer's face.

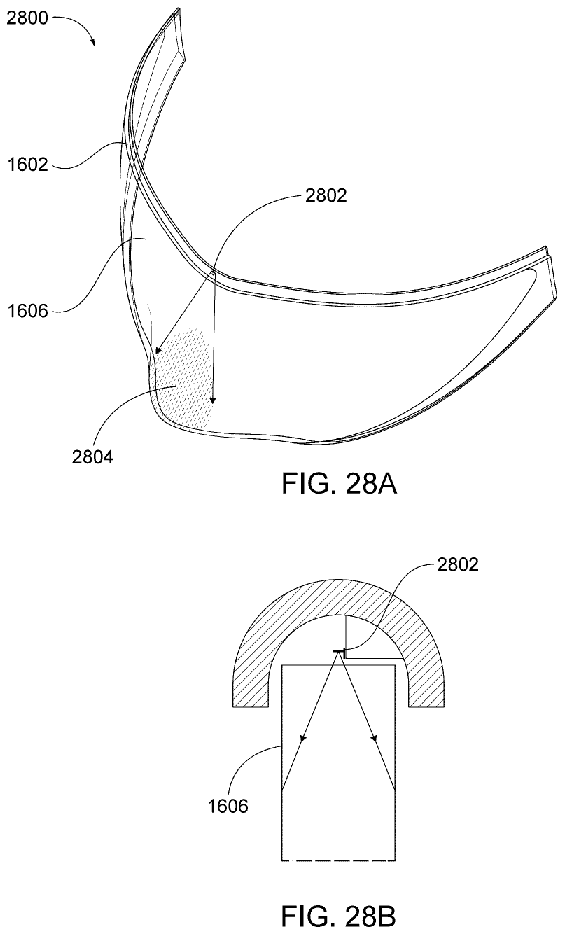

With reference to FIGS. 28A and 28B, an eyewear 2800 according to another embodiment includes a frame 1602 to which a screen 1606 is coupled. In this embodiment, an edge-emitting infrared diode laser 2802 is coupled to the top section of the frame, e.g., it is positioned within a heat-sink channel formed in the frame. The radiation emitted by the infrared diode laser 2802 is introduced into the screen, which functions as a waveguide for transmission of the infrared radiation into lower portions of the screen including a nose-receiving section of the screen. A portion of the outer surface of the nose-receiving section of the screen includes surface texturing 2804 that can cause scattering of the incident radiation such that at least a portion of the incident radiation is directed to the wearer's nose to cause its heating. By way of example, the surface texturing can be characterized by a surface roughness of 1 micron or less (e.g., in a range of about 100 nm to about 1 micron).

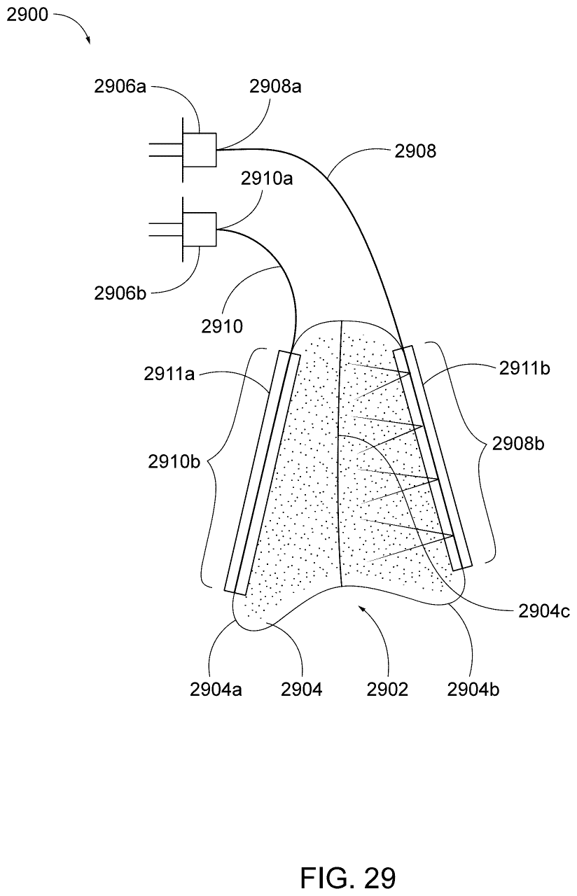

FIG. 29 schematically depicts another embodiment of a nose-heating (also referred to as nose-warming) system 2900 that includes nose-heating element 2902 having a frame 2904, which includes two wing portions 2904a/2904b that are connected by a living hinge 2904c such that when the nose-heating element is worn by a user, the living hinge is at least partially over the bridge of the user's nose and the wing portions can be folded about the living hinge to cover lateral sides of the nose. In this embodiment, the nose-heating system 2900 includes two infrared radiation-generating sources 2906a and 2906b and two optical fibers 2908 and 2910, which are optically coupled at proximal ends thereof 2908a and 2908b to infrared sources 2906a and 2906b, respectively, to receive radiation therefrom, e.g., radiation at a wavelength of about 850 nm. The nose-heating system further includes two cylindrical optical diffusers 2911a and 2911b (such as those disclosed in U.S. Pat. No. 6,270,492, which is herein incorporated in its entirety by reference) in which distal portions 2908b and 2910b of the optical fibers are disposed. Each of the cylindrical optical diffusers is coupled to one edge of the frame of the nose-heating element to deliver radiation into the body of the frame. In general, each of the optical diffusers can be optically coupled to any of available edges of the frame of the nose-heating element. In some embodiments, the unused edges of the frame and/or the optical fibers (or at least the distal portions thereof) can be covered with aluminum-based tapes to make them reflective.

In some embodiments, an infrared-reflecting coating can be disposed on an outer surface of the nose-heating element to redirect the radiation incident thereon onto the face of a user wearing the nose-heating element. While in this embodiment two infrared radiation sources are employed, in other embodiments a single radiation source (e.g., either radiation source 2906a or 2906b) may be employed. In another embodiment a white polymer reflective film is placed on the back of the nose piece screen in lieu of the dielectric coating.

While in the above embodiment the frame includes a hinge, in other embodiments, the frame does not include such a hinge to facilitate the functioning of the frame as a waveguide through which the radiation can propagate.

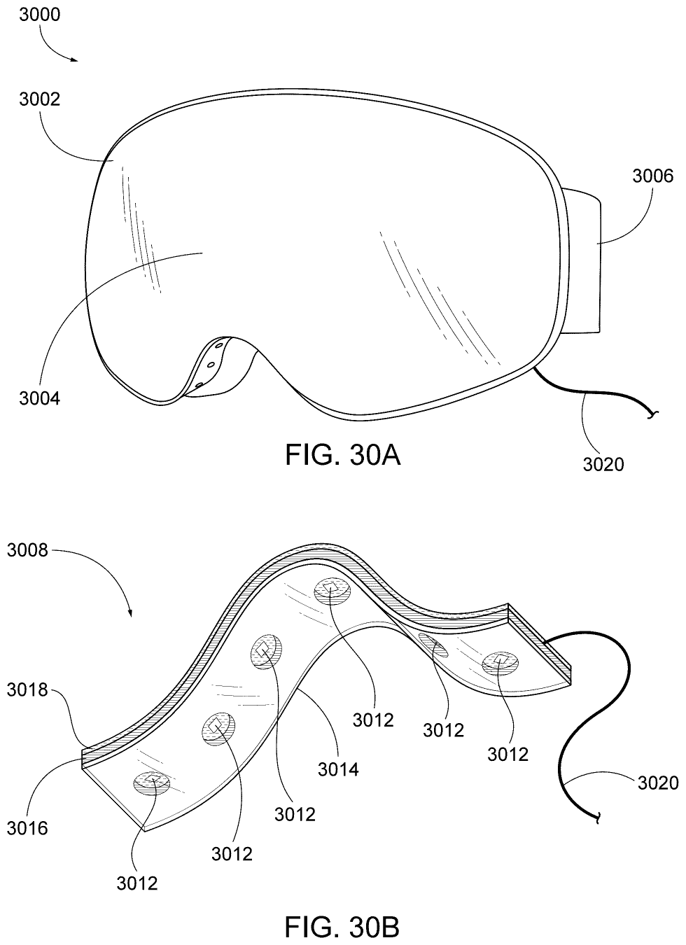

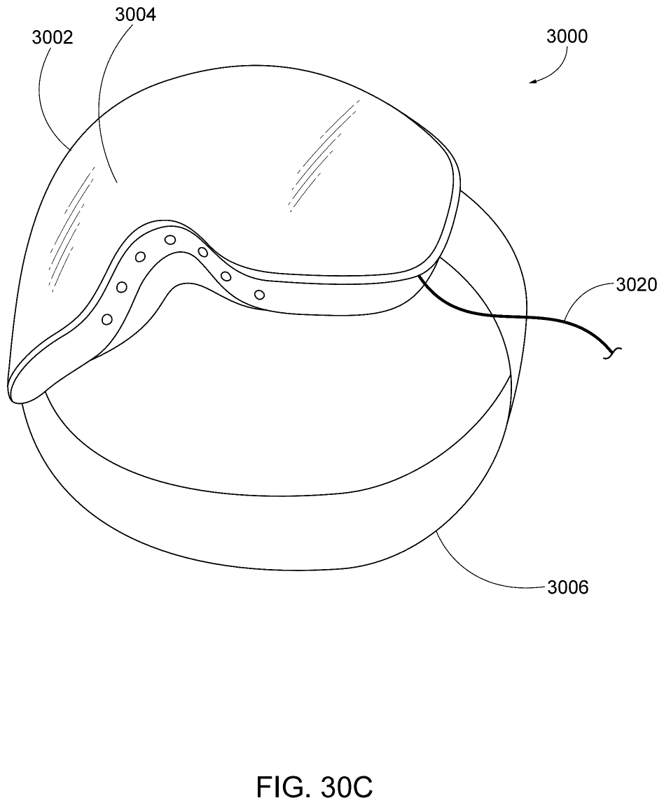

With reference to FIGS. 30A, 30B, and 30C, a goggle 3000 according to an embodiment includes a frame 3002 that defines a field of view through which a wearer can receive light from an external environment. A transparent optic 3004, e.g., formed of a transparent plastic material, is coupled to the frame and extends across the field of view. A strap 3006 releasably coupled to the lateral sides of the frame allows engagement of the frame with the wearer's head.

The goggle 3000 further includes a heating strip 3008 that is coupled to the frame and is shaped and positioned such that it extends over a portion of the wearer's nose when the goggle is worn. More specifically, the heating strip 3008 is curved so as to provide a recess that can receive at least a portion of the wearer's nose. As discussed in more detailed below, the heating strip 3010 includes a plurality of infrared radiation emitting sources 3012 generating infrared radiation that is directed toward the wearer's nose when the goggle is worn.

More specifically, the heating strip 3008 includes a radiation source layer 3014 on an inner surface of which the plurality of radiation sources 3012 are mounted. As discussed further below, in various embodiments, a plurality of conductors can be embedded in the radiation source layer 3014 for supplying electrical power to the radiation sources. Further, the radiation source layer can act as a heat sink for dissipating heat generated by the radiation sources. In some embodiments, a thin metallic layer can be disposed on the outer surface of the radiation source layer 3014 to reflect backscattered radiation towards the wearer's nose.

In this embodiment, the radiation emitting sources 3012 are in the form of six light emitting diodes (LEDs). While in this embodiment, six infrared light-emitting diodes are employed, other numbers of LEDs or other infrared radiation sources (e.g., infrared lasers) can also be employed.

The heating strip 3008 further includes a thermally conductive layer 3016 that is positioned over the radiation source layer 3014 (e.g., in contact with a surface of the radiation source layer on which the radiation sources are mounted), and includes a plurality of openings that are positioned in substantial register with the plurality of radiation sources to allow the passage of radiation emitted from the plurality of radiation sources. By way of example, the thermally conductive layer 3016 can be formed of a suitable thermally conductive polymeric material, such as polyethylene.

In this embodiment, an infrared transparent layer 3018 is positioned over a surface of the thermally conductive layer opposed to the surface of the thermally conductive layer that is in contact with the surface of the radiation source layer. In some embodiments, the layer 3018 can be formed as a radiation diffusing layer having a plurality of radiation scattering centers that are distributed within a matrix. In such embodiments, the radiation diffusing layer can cause diffusion of the radiation passing therethrough, thereby enhancing the uniformity of illumination of a wearer's nose.

In this embodiment, a plurality of electrical conductors 3020 can extend partially through the radiation source layer 3014 and can be partially accessible external to the strip to allow electrically connecting the LED's 3012 to a power source (not shown), such as a battery. In some embodiments, such a battery may be incorporated into the frame of the goggle or can be stored in a compartment coupled to the strap 3006 (e.g., in a manner shown in FIG. 19 above).

In use, when the goggle 3000 is worn by a user, the radiation emitted by the LEDs can illuminate the wearer's nose and cause heating thereof. It has been unexpectedly discovered that not only the radiation emitted by the LEDs but also the heat dissipated by the LEDs can synergistically provide effective heating of a wearer's nose such that a lower electrical power would be needed for powering the LEDs. This can in turn allow the use of a smaller battery for supplying power to the LEDs over a temporal period during which the LEDs can continuously operate to provide heating of the wearer's nose (e.g., about 8 hours).

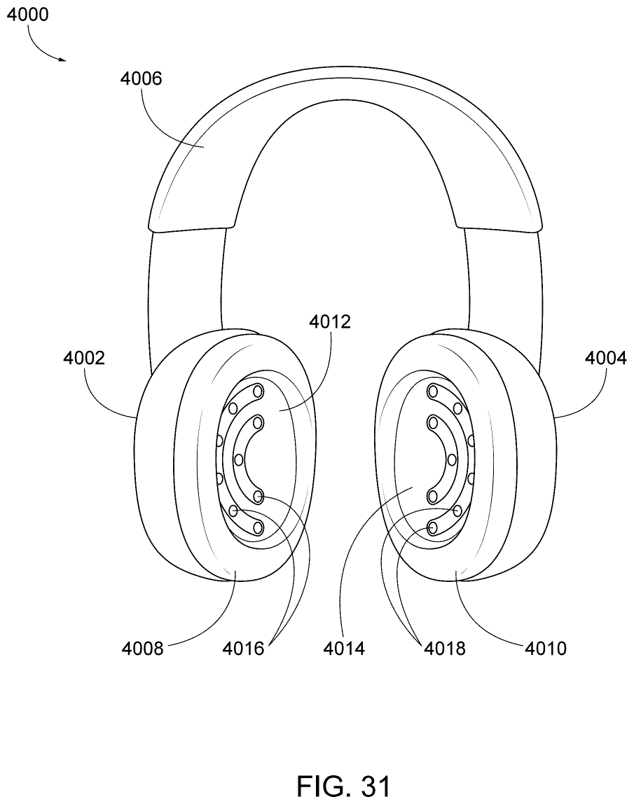

In another aspect, the present teachings provide devices that can be worn by a user and can provide infrared heating of the wearer's ear(s). By way of example, FIG. 31 schematically depicts an ear warming device 4000 (which is herein also referred to as an earmuff or a near infrared earmuff, or its abbreviation a NIR-muff), which includes a right ear heating element 4002 and a left heating element 4004, which are coupled to a hanger 4006. The earmuff 4000 is worn as the hanger 4006 is placed over a user's head. The hanger can be adjustable so as to allow pressing the right and the left ear warming elements 4002/4004 against the user's ears when the earmuff is worn by the user.

In this embodiment, each of the ear warming elements 4002/4004 includes a pad 4008/4010 that is detachably attached to an opening end side of an ear cup 4012/4014. A plurality of infrared emitting radiation sources 4016/4018, such as infrared emitting LED's, are disposed within ear cups 4010/4012. By way of example, the infrared sources can be incorporated in a plurality of strips, each of which can have a structure, e.g., similar to that discussed above in connection with the heating strip 3008, with the strips disposed within the ear cup. While in some embodiments the strip can be fixedly attached to an inner surface of the respective ear cup, in other embodiments it can be detachably attached to that surface. By way of example, and without limitation, an adhesive layer disposed on a back surface of the strip can allow detachable attachment of the strip to the inner surface of the ear cup.

In various embodiments, a power source (not shown), e.g., a battery, for supplying power to each set of the infrared sources 4016/4018 can be incorporated in the body of the respective ear cup 4012/4014, by way of example. In other embodiments, a stand-alone power source that can be readily carried by the wearer of the ear warming device can be employed. Although in this embodiment, infrared LEDs are employed as the infrared emitting sources, in other embodiments other infrared emitting sources (e.g., infrared lasers) may be used.

In various embodiments, the heating of a wearer's ears can be achieved by radiation heating of the tissue together with the waste heat generated by the circuitry of the infrared radiation sources.

Further, in some embodiments, the ear warming device may be configured as a headset including a microphone incorporated in each ear cup.

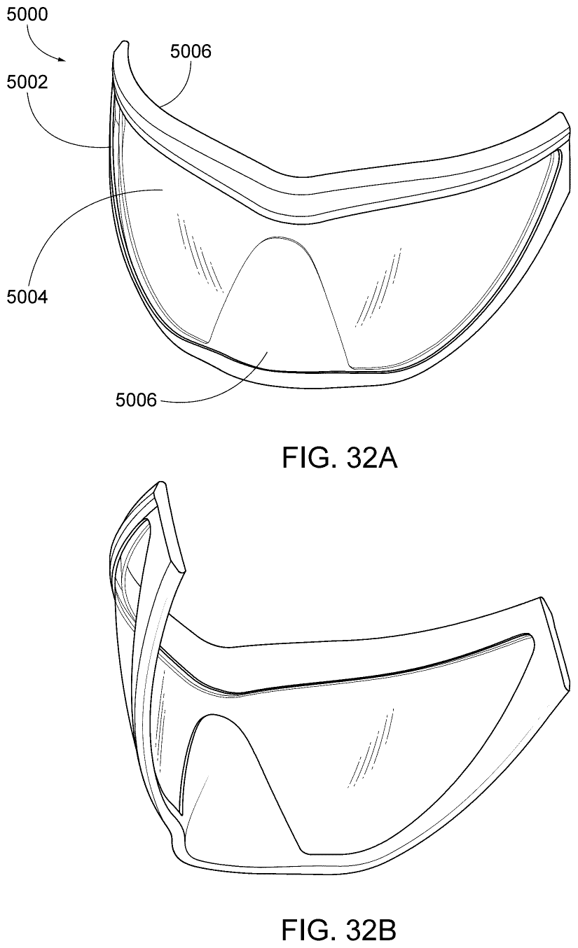

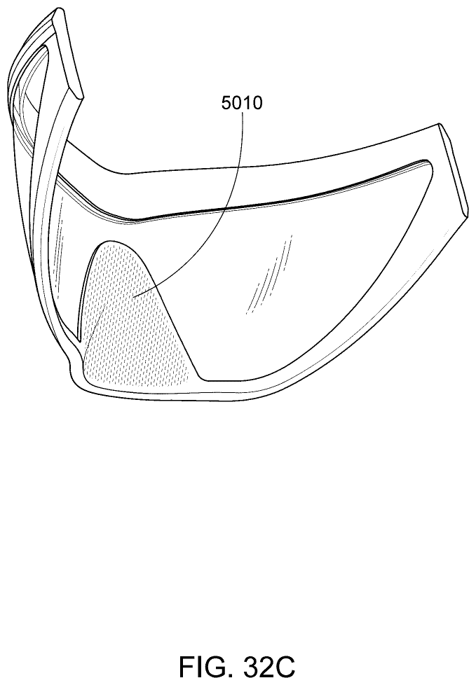

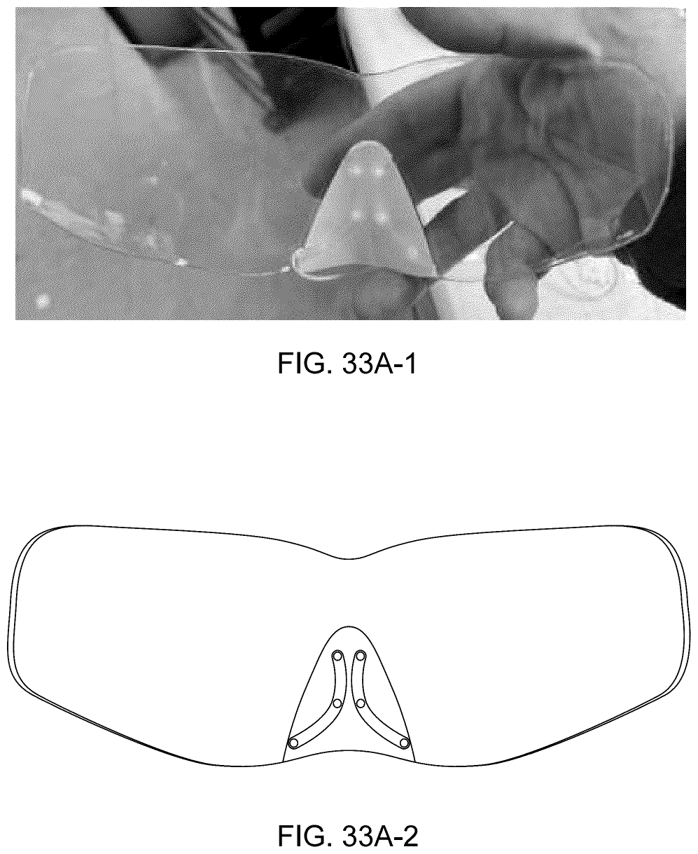

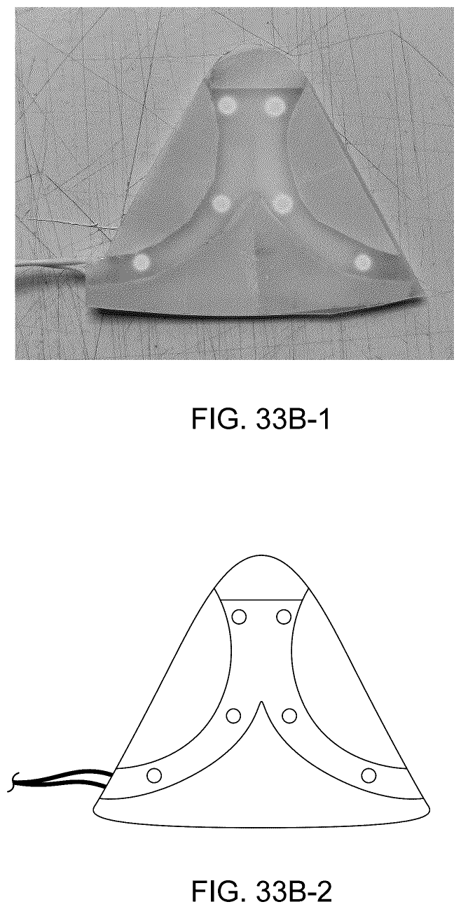

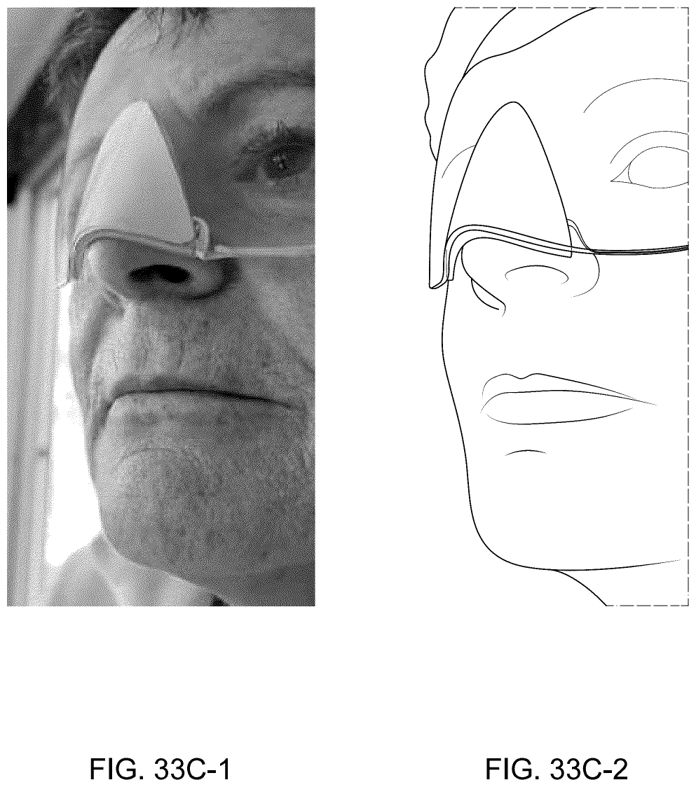

FIGS. 32A, 32B and 32C schematically depict an eye wear 5000 according to an embodiment, which includes a frame 5002 to which a transparent optic 5004 is coupled to allow a user to view an external environment when wearing the eye wear. By way of example, and without limitation, the frame 5002 can be formed from a variety of suitable materials, such as a variety of polymeric, metallic, or a combination of polymeric and metallic materials. Some suitable examples of such materials include, without limitation, polyurethane, nylon, TR90 (a flexible and durable thermoplastic material), Titanium, Aluminum and Beryllium. Further, the transparent optic of the eye wear can be formed of a variety of suitable transparent materials, such as, polycarbonate, Trivex (a highly impact-resistant material), and CR-39 (a scratch-resistant plastic).

The eye wear 5000 further includes an infrared nose warming element 5006 that is either fixedly or detachably attached to the frame 5002. By way example, lateral surfaces of the nose warming element 5006 can be glued to an inner surface of the frame that circumscribes the lateral surface of the nose warming element. The nose warming element is coupled to the frame such that it will be positioned over (and in some embodiments in contact with) at least a portion of the nose of the wearer of the eye wear. Further, the nose-warming element 5006 can include a plurality of infrared radiation emitting sources (such as a plurality of infrared LEDs) that can generate infrared radiation for illuminating the wearer's nose and hence cause warming thereof.

For example, in various embodiments, the nose-warming element depicted in FIG. 3A and described above can be used as the nose-warming element 5006. But it should be understood that the structure of a nose-warming element that can be employed in the eye wear 5000 is not limited to that shown in FIG. 3A and other structures such as those depicted in FIGS. 5A, 5B, 5C, and 6 can also be utilized.

With continued reference to FIGS. 32A, 32B and 32C, in this embodiment, a padding (liner) 5008 is attached to the perimeter of the frame of the eye wear. The padding can be formed of a soft polymeric material, such as a memory foam combined with nylon mesh fabric. In various embodiments, the padding 5008 can provide a soft surface between the goggle frame and the wearer's face to help prevent discomfort from potential pressure points. Further, the padding 5008 can allow a snug fit between the eye wearer's frame and the wearer's face, thereby enhancing stability and preventing slippage of the eye wear, e.g., during certain activities such as skiing or snowboarding. In other embodiments, the eye wear may not include such a padding.

With particular reference to FIG. 32C, in this embodiment, a thin mild adhesive layer 5010 is disposed on a back surface of the nose-warming element (i.e., a surface of the nose-warming element that is in proximity of the wearer's skin) to stabilize the nose-warming element on the wearer's nose. Some examples of suitable adhesives include, without limitation, acrylate monomer adhesives, which can include, e.g., a base monomer (such as methyl methacrylate), functional monomer (such as acrylic acid), and additives (such as plasticizers, tackifiers, and crosslinkers).

In use, when the eye wear 5000 is worn by a user and the infrared radiation sources, e.g., LEDs, associated with the nose warming element are activated, the radiation emitted by the infrared radiation sources can be absorbed by the tissue of the wearer's nose, thereby causing heating thereof. Further, as noted above, it has been discovered that in some embodiments, the waste heat generated by the infrared radiation source can combine in a synergistic way with the heat generated via absorption of the infrared radiation by the wearer's tissue to provide an efficient heating of the tissue. In other words, in various embodiments, heat generated by near infrared radiation emitted by the radiation sources together with far infrared radiation associated with heat dissipated by the infrared source can collectively cause heating of the wearer's tissue, e.g., the wearer's nose.

The following examples are provided for further elucidation of various aspects of the present teachings and is presented only for illustrative purposes.

### EXAMPLES

### Example 1

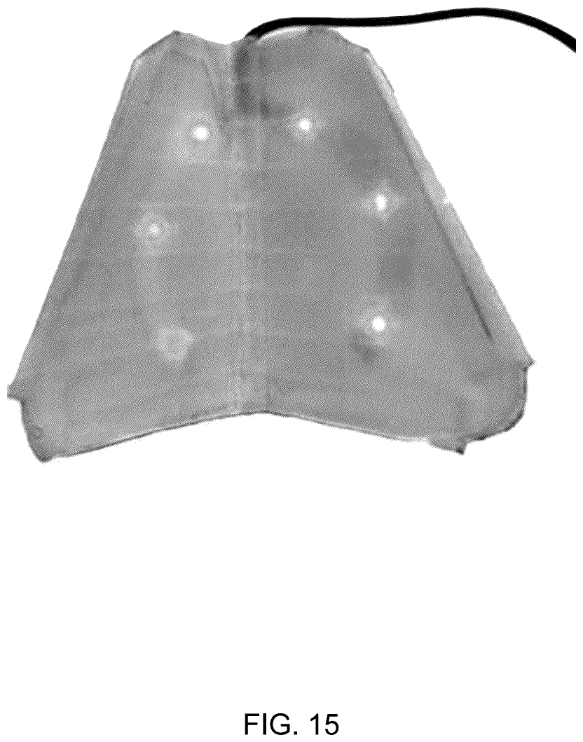

FIG. 15 shows an image of a prototype nose-heating element according to an embodiment, which includes a frame formed of PETG (polyethylene terephthalate glycol). A flat PETG layer was cut and thermally formed to match the nasal bridge. Two curved LED circuits, each having 3 LEDs, were adhered to the frame. A reflective white back film was laminated on the back of the frame. Specifically, adhesive backed Dacron was utilized. A front translucent film, a translucent nylon adhesive backed cloth, was laminated onto the front of the frame that diffuses the radiation from the LEDs and prevents the rough LED circuit from touching the skin. The front layer can also shield the LEDs from dirt and moisture. The layers were connected by sewing, though other techniques such as thermal lamination can also be employed. The two LED circuits were electrically connected in parallel to a DC source. The 3 LEDs in each circuit were electrically connected in series and could be operated with a 5 V lithium ion battery. Though not used in the prototype, an optional thermal inline fuse can be included in the circuits to prevent skin overheating. Each LED was capable of emitting more than 100 mW of radiative power and required about 3 W of DC power. Warming of the nasal passage was easily demonstrated and resultant warmth was quite pleasant.

### Example 2

A prototype radiant nose warmer was constructed and mounted onto a transparent visor. This prototype nose warmer included six LEDs of the size 3535, which were surface mounted onto a curved surface of prefabricated PCB (printed circuit board). The LEDs were spaced relative to one another according to the teachings of aforementioned U.S. Pat. No. 12,053,642.

Three of the LEDs were arranged in series and the series arrangement was extended to the other of the two curved circuits. The application of a required voltage of about 10V to the LEDs connected in series resulted in the flow of a 0.4 A current in the circuit. Each LED emitted radiation at a power of approximately 350-550 mW. A wavelength of 940 nm was chosen due to the high absorption of this wavelength in skin by water.

The curved circuits were adhered to a nose shaped aluminum blank circuit board of about 150 μm thickness by a thermally conductive silicone glue. A thermal pad was then applied to the top surface of the nose shaped circuit. The pad was formed of a thermally conductive material that was about 3-mm thick and was composed of silicone filled with ceramic particles, though metallic particles can also be used. The pad had a light gray color with an adhesive applied to both of its surfaces. The back side of the pad adhered to the circuit board. A translucent PET (polyethylene terephthalate) diffusing film was then applied on top of it, adhering to the front surface. The finished nose pad was then glued to a clear mask in a manner discussed above to fabricate a prototype device according to an embodiment, which could be tested for heating and comfort.

The total IR emission of the 6 LEDs was about 1.8 W@940 nm. In various embodiments, wavelengths longer than the upper end of the human eye's visible spectrum, e.g., longer than about 800 nm, are needed to eliminate eye detection of the emission. The 850 nm-950 nm range is particularly appropriate for this heating application. By design, the entire power applied, which includes both the radiant infrared and waste heat far infrared emissions combined to heat the nose, face or ears. In this prototype, an electrical power of 4.5 W was sufficient to provide a comfortable heating of the nose. At this setting, a 10,000 mAH (milliampere-hour) lithium ion battery capable of running this unit for 6-7 hours before requiring a recharge was employed. A battery of this size can be readily mounted onto a variety of eyewear devices, such as helmets, or be stored elsewhere, e.g., a user's clothing.

Those having ordinary skill in the art will appreciate that various changes can be made to the above embodiments without departing from the scope of the invention.

1. An eyewear, comprising:
a frame configured to be worn by a wearer,
at least one transparent optic coupled to the frame so as to allow the wearer to view an external environment through said at least one transparent optic, and
an infrared nose heating element coupled to the frame so as to be positioned at least partially over the wearer's nose,
wherein said infrared nose heating element includes at least one infrared radiation source for illuminating at least a portion of the wearer's nose, thereby causing heating thereof.

2. The eyewear of claim 1, wherein said infrared nose heating element comprises a shell to which said at least one infrared radiation source is coupled.

3. The eyewear of claim 1, wherein said at least one infrared radiation source is capable of emitting radiation with a wavelength in a range of about 800 nm to about 2 microns.

4. The eyewear of claim 1, wherein said at least one infrared radiation source comprises a plurality of infrared radiation sources incorporated in a strip attached to said shell.

5. The eyewear of claim 1, wherein said at least one infrared radiation source comprises an infrared light emitting diode (LED).

6. The eyewear of claim 1, wherein said at least one infrared radiation source comprises an infrared laser.

7. The eyewear of claim 1, wherein said at least one infrared radiation source generate infrared radiation at a power in a range of about 100 mW to about 5 W.

8. The eyewear of claim 1, further comprising a padding attached to a perimeter surface of the frame of the eyewear.

9. The eyewear of claim 7, wherein said padding comprises a soft polymeric material.

10. The eyewear of claim 8, wherein said soft polymeric material comprises a memory foam combined with a nylon mesh fabric.

11. The eyewear of claim 9, further comprising a mild adhesive layer disposed on a surface of said shell to stabilize said shell on the wearer's nose.

12. The eyewear of claim 1, wherein said infrared nose heating element is fixedly attached to the frame.

13. The eyewear of claim 1, wherein said infrared nose heating element is detachably attached to the frame.

14. The eyewear of claim 1, wherein said infrared nose heating element comprises a thermally conductive layer to facilitate transfer of waste heat generated by electric circuitry of said at least one infrared radiation source to the wearer's nose.

15. The eyewear of claim 1, wherein said optic comprises a transparent polymeric material.

16. The eyewear of claim 15, wherein said transparent polymeric material comprises a transparent plastic.

17. The eyewear of claim 1, wherein said optic comprises any of polycarbonate.

18. The eyewear of claim 1, wherein said frame comprises two temples configured for resting on ears of the wearer such that at least each of the wearer's eyes can view the external environment through a respective portion of the optic.

19. The eyewear of claim 17, wherein said at least one transparent optic comprises a unitary optic extending across the wearers face.

20. The eyewear of claim 18, wherein said at least one transparent optic comprise two separate transparent optics each of which is positioned in front of one eye of the wearer when the eyewear is worn by the wearer.

21-52. (canceled)
